(19) United States
(12) Patent Application Publication
(10) Pub. No.: US 2017/0172406 A1
(43) Pub. Date: Jun. 22, 2017

(54) Methods and Apparatus for Optical Controller

(71) Applicant: EyeNetra, Inc., Somerville, MA (US)

(72) Inventors: Vitor Pamplona, Somerville, MA (US); Matthias Hofmann, Allston, MA (US); Nathaniel Sharpe, Cambridge, MA (US)

(21) Appl. No.: 15/127,408

(22) PCT Filed: Mar. 24, 2015
(86) PCT No.: PCT/US15/22138

§ 371 Date: Sep. 19, 2016

Related U.S. Application Data
(60) Provisional application No. 62/103,062, filed on Jan. 13, 2015.
(60) Provisional application No. 61/970,032, filed on Mar. 25, 2014.

(51) Int. Cl. A61B 3/028; A61B 3/113; A61B 3/00

(52) U.S. Cl. CPC A61B 3/028; A61B 3/0025; A61B 3/005; A61B 3/113

(57) ABSTRACT

In illustrative implementations of this invention, a human user mechanically moves one or more moveable parts in a handheld controller, and thereby optically controls a mobile computing device. In illustrative implementations, the optical control is implemented as follows: A camera onboard the mobile computing device captures images. The images show the motion of the moveable parts in the handheld controller. A camera onboard the mobile computing device analyzes these images to detect the motion, maps the motion to a control signal, and outputs a control signal that controls a feature or operation of the mobile computing device.

### RELATED APPLICATIONS

This application is a non-provisional of, and claims the priority of the filing date of, U.S. Provisional Patent Application No. 61/970,032, filed Mar. 25, 2014 (the “032 Application”), and of U.S. Provisional Patent Application No. 62/103,062, filed Jan. 13, 2015 (the “062 Application”). The entire disclosures of the 032 Application and the 062 Application are incorporated herein by reference.

### FIELD OF TECHNOLOGY

The present invention relates generally to control apparatus.

### SUMMARY

In illustrative implementations of this invention, a human user mechanically moves one or more moveable parts in a handheld controller, and thereby optically controls a mobile computing device (MCD). In illustrative implementations, the optical control is implemented as follows: A camera onboard the MCD captures images. The images show the motion of the moveable parts in the handheld controller. A camera onboard the MCD analyzes these images to detect the motion, maps the motion to a control signal, and outputs a control signal that controls a feature or operation of the MCD.

In some implementations of this invention, the mobile computing device (MCD) comprises a smartphone, cell phone, mobile phone, laptop computer, tablet computer, or notebook computer.

The handheld controller includes one or more moveable parts that undergo mechanical movement, relative to the controller as a whole. For example, some of the moveable parts comprise I/O devices (e.g., buttons, dials and sliders) that a human user touches. Other moveable parts comprise parts that are not directly touched by a human user, but undergo mechanical motion, relative to the controller as a whole, that is actuated (e.g., through gears, linkages, or other motion transmission elements) by movement of the I/O devices.

For example, in some cases, a human user rotates a dial on the handheld controller. In some cases, the dial is the pinion in a rack and pinion, such that rotation of the dial actuates linear motion of a rack inside the handheld controller.

A camera in the MCD captures visual data regarding all or part of these moveable parts, while (optionally) one or more light sources in the MCD illuminate the MCD. A computer in the MCD analyzes this visual data to compute position or motion of moveable parts. Based on the computed position or motion of moveable parts, the computer outputs control signals to control operation of the MCD. For example, in some cases, the control signals control light patterns that are displayed by the MCD.

In many implementations of this invention: (1) the handheld controller does not include any electronics, motor, engine or other artificial actuator; and (2) the handheld controller does not have a wired electrical connection to the MCD. As a result, in many implementations, the handheld controller is very inexpensive to manufacture. For example, in some cases the handheld controller comprises plastic, with no electronics.

Advantageously, the handheld controller allows a human user to input complex commands to a MCD by simple mechanical motions. This is particularly helpful at times when all or a portion of the MCD's display screens are being used for another function (such as testing for optical aberrations of a human eye or cataracts) and are not available as a graphical user interface.

In some implementations, the controller is used to optically control an MCD, while the controller and MCD are attached to each other, and a screen onboard the MCD outputs images that are viewed by the human user as part of an eye test (e.g., a test for refractive aberrations of the user's eyes).

The description of the present invention in the Summary and Abstract sections hereof is just a summary. It is intended only to give a general introduction to some illustrative implementations of this invention. It does not describe all of the details and variations of this invention. Likewise, the description of this invention in the Field of Technology section is not limiting; instead it identifies, in a general, non-exclusive manner, a field of technology to which exemplary implementations of this invention generally relate. Likewise, the Title of this document does not limit the invention in any way; instead the Title is merely a general, non-exclusive way of referring to this invention. This invention may be implemented in many other ways.

### BRIEF DESCRIPTION OF THE DRAWINGS

FIGS. 1A and 1B each show a handheld controller that is attached to a mobile computing device (MCD) and that is being held by a human user. In FIG. 1A, the user is holding the controller away from the face. In FIG. 1B, the user is holding the controller up against the user's eyes.

FIGS. 2A and 2B are each an exploded view of a handheld controller and MCD to which the controller is attached. In FIG. 2A, the handheld controller has a single viewport for both of the user's eyes. In FIG. 2B, the handheld controller has two holes, one hole for each of the user's eyes.

FIG. 3 is a block diagram of a handheld controller and MCD.

FIG. 4 shows a perspective view of a handheld controller, including a control canvas that is imaged by a camera in the MCD.

FIGS. 5A, 5B, and 5C each show light emitted from a light source in a MCD, which light strikes a reflector in the controller, and reflects back to a camera in the MCD. In FIG. 5A, the reflector is specular. In FIG. 5B, the reflector is a retroreflector. In FIG. 5C, the reflector is diffuse.

FIGS. 6A, 6B and 6C show three examples of illumination patterns displayed by a MCD display screen to illuminate the handheld controller. In FIG. 6A, the entire display screen is used as a light source to illuminate the handheld controller. In FIG. 6B, a portion, but not all, of the display screen is used as a light source. In FIG. 6C, a region of the display screen used as a light source varies over time.

FIG. 7 is a conceptual diagram that shows steps in a method for using a handheld controller to control operation of an MCD.

FIGS. 8A and 8B are conceptual diagrams, showing examples in which a mapping function is used to calculate the position of a control feature relative to a path. In FIG. 8A, the mapping function is non-periodic. In FIG. 8B, the mapping function is periodic.

FIGS. 9A, 9B, 9C and 9D show the use of calibration features to determine position and path of control features. In FIG. 9A, two calibration features are used, one at each end of a path. In FIG. 9B, a single calibration feature demarks a central point, in the center of a rotational path. In FIG. 9C, multiple calibration features that are offset from each other together indicate the position of a central point. In FIG. 9D, a calibration feature is co-located with the entire path.

FIGS. 10A, 10B, 10C together show steps in a method for determining the path of a control feature, in a noisy image. In FIG. 10A, the light from the MCD comprises a broad visible spectra. In FIG. 10B, the light from the MCD is primarily a first color, thus emphasizing calibration features that reflect primarily light of the first color. In FIG. 10C, the light from the MCD is primarily a second color, thus emphasizing control features that reflect primarily light of the second color.

FIGS. 11A, 11B, 11C and 11D are four views of a face-fitting portion of the controller. The face-fitting portion is configured to be pressed against at least the forehead and cheeks of human user. FIG. 11A is a perspective view; FIG. 11B is a top view; FIG. 11C is a back view; and FIG. 11D is a side view.

FIGS. 12A, 12B, 12C and 12D are four views of an attachment mechanism for attaching the controller to a mobile computing device. FIG. 12A is a perspective view; FIG. 12B is a bottom view; FIG. 12C is a back view; and FIG. 12D is a side view.

FIG. 13A shows a system comprising a machine-readable medium and a handheld controller. FIG. 13B shows examples of locations for a machine-readable medium.

FIGS. 14A, 14B, 14C, 14D, 14E, 14F, 14G, 14H, 141, 14J, 14K, 14L, 14M, 14N, 14O, 14P, 14Q, 14R, 14S, and 14T each show a different example of one or more calibration features that are indicative of a path of a control feature.

FIG. 15 shows an example of concentric rings around eyeports.

FIG. 16 shows an example of relay optics in a controller.

FIG. 17 is a block diagram of a controller device and MCD.

The above Figures show some illustrative implementations of this invention. However, this invention may be implemented in many other ways. The above Figures do not show all of the details of this invention.

### DETAILED DESCRIPTION

In illustrative implementations, a handheld controller is used to control operations of a mobile computing device to which the handheld controller is releasably attached. The handheld controller includes a set of mechanical user interfaces, such as buttons, scroll wheels, knobs, ratchets, sliders and other mechanical components. The handheld controller also includes a set of visual features that are either on, or part of, moveable parts. These moveable parts are either the mechanical user interfaces or components that are mechanically actuated by movement of the mechanical user interfaces. A camera in a mobile computing device is used to detect position or motion of the visual features. Based on this position or motion data, a computer onboard the MCD outputs signals to control operation of the MCD, including the graphics of the device display.

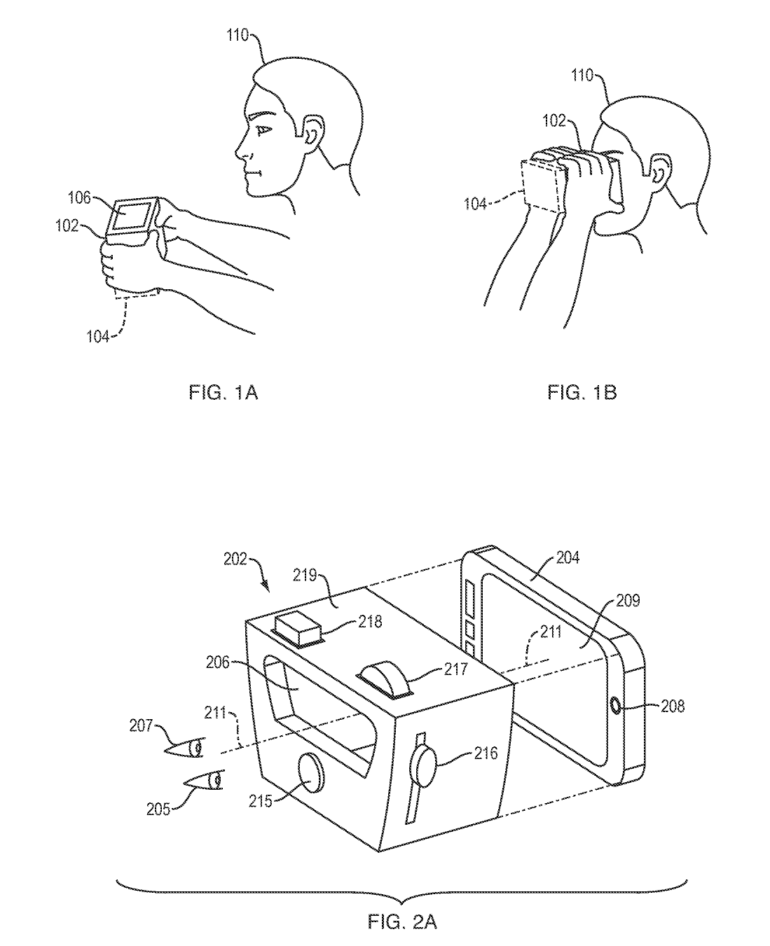

In the examples shown in FIGS. 1A and 1B, a handheld controller 102 is releasably attached to a mobile computing device (MCD) 104. A user 110 controls one or more features or functions of the MCD via the attached handheld controller. The handheld controller includes with one or more user interfaces that are accessed and manipulated by the user's fingers, palms, or wrists. Through the manipulation of the user interfaces, the user controls one or more features or functions of the MCD, including visual content of one or more built-in MCD displays.

In the example shown in FIG. 1A, the user holds the controller away from the face. In the example shown in FIG. 1B, the user holds the controller to his head or in front of his eyes, while viewing the MCD's screen through the viewing port of the controller. Depending on the particular implementation, the controller may vary in size and shape. Preferably, the size and shape of the controller are such that user may freely move the controller to face level. In FIG. 1B, a user views the display and other elements of the MCD. The user does so by looking through a window or view port 106 in the MCD that gives visual access to the inside of the system (e.g., to a display screen in the MCD).

The user holds the controller in one or both hands during operation. In some use scenarios, the user holds the controller with one hand, and uses the other hand to manipulate user interfaces. In some use scenarios, the user uses both hands for securely holding the controller while simultaneously using both hands to manipulate user interfaces. In some use scenarios, the user holds the controller in one hand, while manipulating interfaces with the same hand. In some use scenarios, the controller is be held by one person and controlled simultaneously by a second person (e.g. the second person manipulates the mechanical user interfaces of the controller).

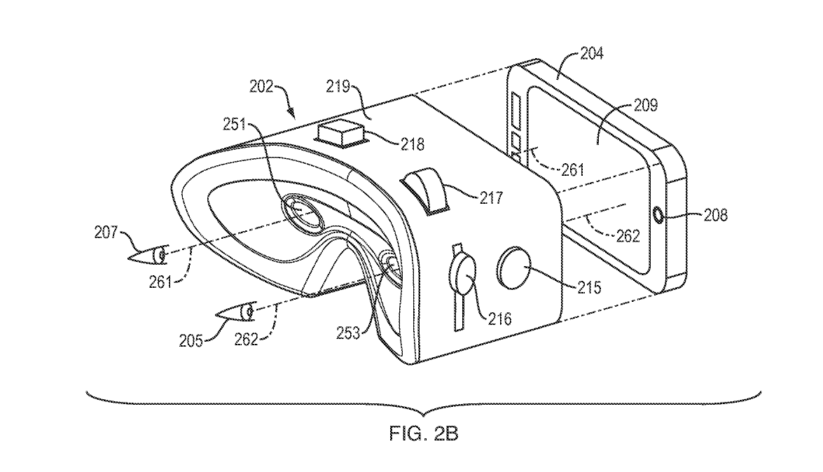

FIGS. 2A and 2B are each an exploded view of a handheld controller and MCD to which the controller is attached. In FIG. 2A, the handheld controller has a single viewport for both of the user's eyes. In FIG. 2B, the handheld controller has two holes 251, 253, one hole for each of the user's eyes.

In some cases, MCD 204 comprises a cellular phone (e.g. a smart phone). The MCD 204 includes a built-in camera or light sensor 208.

The handheld controller 202 includes a housing 219. In addition, the handheld controller 202 also includes mechanical user interfaces that the user manipulates. For example, in some cases, the user interfaces include turn dials 215, sliders 216, wheels 217, or buttons 218.

The handheld controller also includes an attachment mechanism that (a) easily attaches an MCD to the handheld controller, and (b) easily releases the MCD from the handheld controller. Over the course of the handheld device's useful life, the handheld controller is repeatedly attached to, and then detached from, an MCD. During times when the MCD is attached to the handheld controller via the attachment mechanism, the position of the handheld controller relative to the MCD is fixed. The handheld controller includes a window 206 through which a user views a display screen 209 of the MCD, when the controller 202 and MCD 204 are attached to each other.

In the exploded views of FIGS. 2A and 2B, handheld controller 202 and MCD 204 appear to be separated from each other. However, in actuality, when controller 202 and MCD 204 are attached to each other, MCD 204 is touching controller 202.

In FIG. 2A, an opening or hole 206 passes through the controller 202. A line-of-sight 211 passes through the opening 206 and extends to a screen 209 of the MCD, when the MCD 204 and controller 202 are attached to each other.

In FIG. 2B, the user's right eye 205 looks through hole 253, and the user's left eye 207 looks though hole 251. Lines-of-sight (261, 262) pass through the holes 251, 253, and extend to a screen 209 of the MCD, when the MCD 204 and controller 202 are attached to each other.

Thus, in FIGS. 2A and 2B, a view extends through the controller 202 such that at least a portion of a screen 209 of the MCD 204 is visible from where eyes 205, 207 of a human are located, when the MCD 204 and controller 202 are attached to each other and a surface of the controller is pressed against the forehead and cheeks of the human.

Depending on the particular implementation, a variety of different attachment mechanisms are used to releasably join the controller 202 and MCD 204 together. For example, in some cases, an attachment mechanism that is part of the handheld controller 202 comprises: (1) a clip that clips over the MCD; (2) one or more flexible bands or tabs that press against the MCD; (3) retention features that restrain the MCD on at least two edges or corners of the MCD (including retention features that are part of an opening in the controller); (4) a slot, opening or other indentation into which the MCD is wholly or partially inserted; (5) a socket into which the MCD is partially or wholly inserted into the controller; (6) a door or flap that is opened and closed via a hinge, which door or flap covers a socket or indentation into which the MCD is inserted; (7) a mechanism that restrains motion of the MCD, relative to the controller, in one or more directions but not in other directions; (8) a mechanism (e.g., a “snap-fit”) that snaps or bends into a position that tends to restrain motion of the MCD relative to the controller; or (9) one or more components that press against MCD and thereby increase friction and tend to restrain motion of the MCD relative to the controller.

A human user employs the mechanical interfaces of the controller to optically control one or more features or functions of the MCD, including to control MCD functions, to trigger device events, to launch or control applications that run on the MCD, or to display animated graphics on the MCD's display screen. A computer onboard the MCD recognizes mechanical interfaces that are in the handheld controller and in the camera's field of view, links a change in interface position to an applied user action, and generates control commands. For example, in some cases, a wheel is rotated over a given time period, and the camera detects the relative or absolute displacement of the wheel (e.g. an angular change) and then generates a command that is subsequently executed.

In illustrative cases, an optical link is established through light interactions between the controller and the MCD. A light source, originating from the MCD, illuminates the mechanical user interfaces, which subsequently reflect a portion of the original light back to the MCD. The reflections are recorded by one or more light sensors on the MCD, such as a CCD camera. A computer onboard the MCD analyzes the recorded light signals (e.g., to determine light source shape or intensity), and based on data regarding these light signals, generates control signals that are subsequently executed by the MCD.

In many implementations, the MCD is attached to the controller such that a display or other light source on the MCD faces towards user interfaces of the controller. In some cases, a secondary display or light source is present on the MCD, and, when the MCD and controller are attached to each other, one display or light source on the MCD faces outwards to serve as a graphical or visual user interface for interacting with human users, and the second display or light source on the MCD faces the user interfaces and serves as a controllable light source to illuminate the controller.

In the example shown in FIG. 2, the MCD display faces toward the inside of the handheld controller and serves a dual purpose: it acts as a light source to illuminate or code user interfaces, as well as a display for outputting information in the form of graphics. The display light source output is either constant over a given period, time-varying, or spatially varying. In some cases, color output capabilities of the MCD display are used within a given constant or time-varying sequence. The MCD display selectively illuminates the mechanical user interfaces of the controller to create unique reflection signal characteristics that correspond to a specific user input command. In addition, in some cases, other light parameters such as phase and polarization are also used to amplify the distinction between user interface states of the controller. In some cases, other light sources from the MCD are used alone or in conjunction with the display to control the sources via MCD software.

In illustrative implementations, built-in light sensors in the MCD capture positional information regarding the mechanical user interfaces. For example, in some cases, a built-in camera is used to record light that reflects from the user interfaces. The camera includes one or more lenses and is located on the front or back of the MCD. In some cases, other sensors (such as accelerometers, illumination sensors, proximity sensors, or single-pixel detectors) are utilized in the system. In some cases, the camera or light sensors include electronic circuits, optical components, and embedded software to aid image capturing and processing functions.

In the example shown in FIG. 2, the handheld controller is a hollow structure. The user interfaces on the outside surface are accessible to a human user. The user interfaces have mechanical subcomponents that are not visible from outside the controller, but partially or fully visible by the camera or light sensors of the device. In some cases, when the controller is attached to the MCD, the controller envelops a part, or all, of the MCD such that the only light striking a screen of the MCD is light emitted by the MCD itself and reflected back to the MCD.

In some embodiments, the handheld controller has slots or openings such that ambient light enters and acts as an alternative or enhancing light source. Similarly, in some cases, the controller has larger openings, such that some or all of the user's fingers fit through them. In that case, user interfaces are located on the inside of the hollow controller for the user to access and control. In some cases: (a) the handheld controller is structurally minimal with sufficient structural support to hold the mechanical user interfaces and the MCD in place; and (b) ambient light is present inside the controller, even when the controller is attached to the MCD through a rigid physical connection.

In some cases, the handheld controller is manufactured from one or more lightweight and or biodegradable plastics. In many embodiments, the controller contains no electronic or metal components and is constructed entirely from plastic through molding techniques or 3D-printing systems.

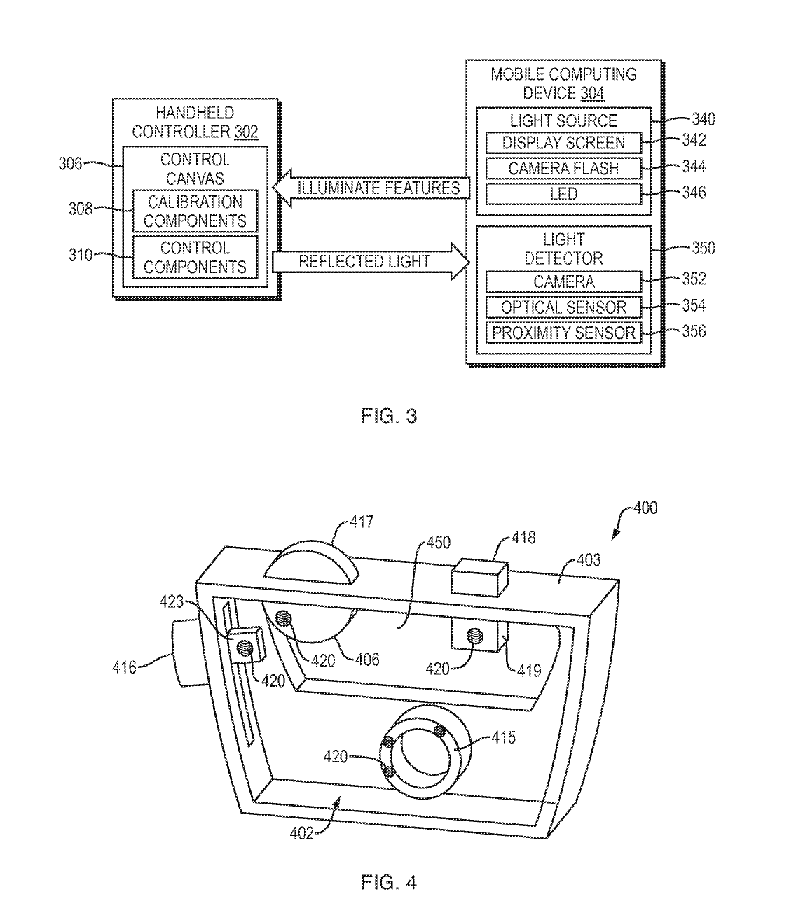

FIG. 3 is a block diagram of a handheld controller 302 and mobile computing device 304. The MCD is controlled by the controller through an optical control method. In this method, a light source 340 emits light that illuminates a control canvas 306. A portion of the light reflects from the control canvas and travels to, and is detected by, a light detector 350. In many cases, both the light source and the light detector are part of an MCD, and the control canvas is part of a handheld controller. The MCD includes one or more light sources, such as a display screen 342, a camera flash 344, or an LED 346. The control canvas in the handheld controller 202 includes control components 310 that are connected to the mechanical user interfaces 215-218. The control components 310 (and visual features affixed to the control components) are moveable relative to each other and to the housing of the handheld controller. In some cases, the control canvas also includes calibration components 308 that facilitate visual detection of the control components 310. The calibration components 308 have a fixed position (i.e., are not moveable) relative to each other and to the housing of the handheld controller. Thus, visual features affixed to the control components 310 are moveable relative to the housing of the handheld controller; and visual features affixed to the calibration components 308 are not moveable relative to the housing of the handheld controller. Built-in light sensors of the MCD are used to visually detect the control components of the handheld controller. Light detectors in the MCD comprise one or more cameras 352, optical sensors such as single pixel detectors (e.g. ambient light sensor) 354, or proximity sensors 356.

In the example shown in FIG. 3, a method includes at least the following steps: Light is emitted from the light source of the MCD in the direction of the control canvas. Then, the light is both absorbed and reflected by the components in the control canvas giving a specific spatial light signature at a given time point. Then, the reflected light from the control canvas is captured by a light sensor on the MCD. This sequence is repeated continuously, in periodic time intervals or in random time intervals. Alternatively, the sequence is performed only once per system application.

In many cases, the fastest repetition rate of the method is defined by the component with the slowest operational rate. In some cases, for example, the slowest operational rate is: (a) the frames-per-second output of a graphical display; (b) a delayed mechanical response of components in the control canvas to physical motion applied by the user; or (c) a frames-per-second rate at which control signals are detected by the camera.

In some cases, a system (which comprises the MCD and controller) operates with a given set of initial parameters that are defined prior to the system application. In some cases, the system also changes parameters of certain components and their subcomponents during runtime dynamically or through feedback from another component's reading. For example, in some cases, parameters of the light source include the intensity of the emitted light, the rate of light output (e.g. rapid or varying on/off light triggers), spatial coding, or a combination of all within a given time interval. The color output is defined a priori or varied during runtime. In some cases, parameters of the light detector include the rate of capture (frames per second), the sensitivity of the detector during each light capture interval, or the sensitivity to a given color (i.e. wavelength).

FIG. 4 shows a perspective view of a handheld controller, including parts of the controller that are seen by a camera in the MCD. In FIG. 4, the handheld controller 400 includes a control canvas 402. The control canvas 402 is visible from the vantage point of a camera in the MCD. The control canvas 402 comprises components (e.g., 406, 415, 419, 423) that are attached to the mechanical user interfaces. In the example shown in FIG. 4, a rotating component 415 is connected to an external dial interface, a sliding plate 423 is connected to an external linear slider 416, a gear connects rotator 406 and an external dial 417, and a lever 419 is connected to the external button 418. A viewport 450 comprises a window for the user to peer through and see the MCD display. In some cases, the window includes a glass or plastic separator fitted with optical lenses. The handheld controller 400 includes a housing 403 that supports the control canvas 402. In some cases (such as the example shown in FIG. 4), the housing 403 at least partially surrounds the control canvas 402.

Visual features 420 are affixed to, or are part of, components in the control canvas, including: (a) one or more components that are moveable relative to the housing of the controller, and (b) one or more components that have a fixed position relative to the housing of the controller. A visual feature 420 that is placed on a moveable component moves when that component moves, and thus facilitates motion tracking of that component.

The camera of the MCD images the control canvas of the handheld controller. Preferably, the MCD is attached to the controller so that the field-of-view of the camera coincides with the control canvas area. In some cases, the control canvas is partially occluded from the camera field-of-view. Mechanical manipulation (by a human user) of a user interface onboard the handheld controller causes the orientation or position of one or more of the visual features to change over a given time period. This causes a spatial and temporal change in the control canvas's layout, effectively changing the visual content, which the camera of the MCD records. A computer determines the visual content of the control canvas by the instantaneous location of the user interfaces, canvas elements, and their corresponding visual features. In some cases, the computer detects “background” areas in the control canvas that are void of visual features used for control.

In the example shown in FIG. 4, a component in the control canvas is connected to an external I/O device onboard the controller, such that physical displacement of external I/O device causes a positional change in a visual feature (control feature) affixed to that component. A camera tracks the positional change, maps the positional change to a control command, and outputs a control command. For example, when external button 418 is pressed by a human user, this causes a corresponding movement of lever 419. A camera tracks this movement of lever 419, maps this movement to a control command, and outputs a control command. In many cases, visual features are visible on the canvas. In other cases, visual features are partially or fully occluded, and are revealed before, during, or after a user's interaction on a user interface.

In illustrative implementations, visual features 420 are either control features or calibration features. Control features are located on components that are moved by the mechanical user interfaces, which are in turn mechanically moved by human input (e.g., pressing a button, sliding a linear slider or turning a dial). A computer analyzes a camera image stream in order to track motion of the control features, maps the motion to control signals, and outputs control signals to modify a graphic display of the MCD in real time. Thus, mechanical movement of a user interface of the handheld controller causes real-time changes in the graphic display onboard the MCD. A computer onboard the MCD performs a frame-by-frame analysis of feature movements in the camera images. The computer calculates relative control feature displacements, color variances, or intensity changes that occur over time in the frames captured by the camera. The computer recognizes features and detect changes in spatial pixel intensity from the camera's available monochrome or color channels. The computer tracks spatial displacement or variation of each feature, including linear or rotational displacement, or changes in the spatial size (area) or relative separation of the feature.

In illustrative implementations, calibration features are used for calibration procedures, positional reference, signal quality check, or device recognition. The calibration features are located on components that either have a fixed or moving position, relative to the controller housing. The position of calibration features within the control canvas are related to the position of control features and serve as “anchor” points in order to compute the relative differences between control and calibration features.

Calibration features are desirable, in order to calibrate for physical variations in the controller or MCD, including variations that occur during initial fabrication or during use. For example, in some cases, calibration features are used to accommodate: (a) variations in MCD placement relative to the controller after attaching the controller to the MCD; (b) variations that occur during operation due to mechanical shock or hardware deformation from the user input; or (c) differences in camera optics between MCD models, and resulting differences in image frame content and orientation. In illustrative implementations, calibration features provide visual cues regarding the relative position, orientation, path, or area in which to track control features.

In some cases, a computer analyzes the camera frames and determines position of control features and calibration features relative to each other or relative to the controller itself For instance, in some cases: (a) a rotary dial is a component of a linear slider, such that linear position of the rotary dial varies according to the position of the linear slider; and (b) displacement of the linear slider is detected prior to analyzing rotation of the rotary dial.

In some cases, the material and color of the visual features (e.g., control features or calibration features) facilitate optical tracking. In some cases, the signal strength of visual features in images recorded by the MCD camera is a function of the feature's material composition. Preferably, the contrast between a visual feature and the surrounding background is maximized. For example, in some cases, contrast is enhanced by using materials such as retroreflectors, mirrors, or metallics.

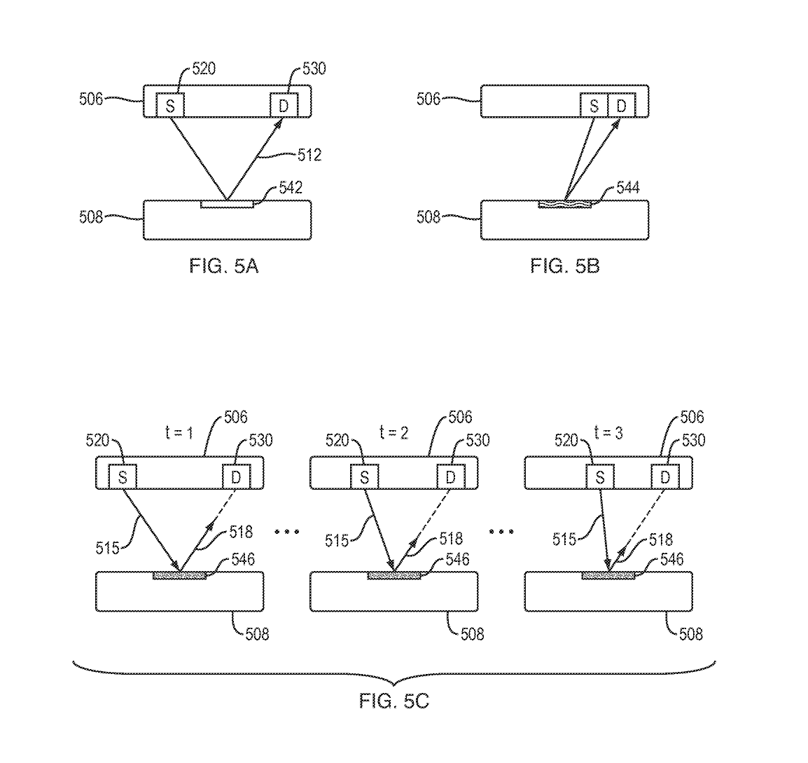

In some cases, material properties of a visual feature are selected such that the visual feature reflects light in a desirable way. This is illustrated in FIGS. 5A, 5B and 5C. In the examples shown in FIGS. 5A, 5B and 5C, the system comprises an MCD 506 and a controller 508. The controller 508 includes a control canvas 402 that includes one or more visual features. The MCD includes a light source 520 and a light detector 530.

In FIG. 5A, visual feature 542 comprises a specular reflector such as a mirror or metallics. This causes light to reflect off the feature at an angle identical to the incoming angle.

In FIG. 5B, visual feature 544 comprises a retroreflective material, such that the light path 512 is predominantly reflected back toward its source. In the example shown in FIG. 5B, the light source and light detector are in close proximity such that the light signal from a retroreflective feature is significantly enhanced while light from other source locations is suppressed. In some cases, visual features comprise concave parabolic mirrors or metallics that redirect light back to the camera, regardless of the location of the light source.

In FIG. 5C, visual feature 546 comprises a diffuse material that reflects incoming light at multiple outgoing angles. For such materials, the light intensity of a given reflection angle may be described by a bidirectional reflectance distribution function (BRDF). In FIG. 5C, the length of arrow 518 symbolically represents the intensity of reflected light that reflects in the direction of arrow 518 (which intensity is specified by the BRDF). Arrow 515 indicates a direction of incident light, which is reflected in direction 518 after it hits the diffuse visual feature 546. In FIG. 5C, light source 520 moves such that it is in different positions at different times (e.g., time t=1, time=2, and time t=3). By moving the position of the light source (i.e. t=1, t=2, t=3), the BRDF skews accordingly, leading to a change in intensity of the reflected light heading to the light detector. In some cases, the BRDF of a visual feature is achieved by selecting the appropriate diffusive surface (e.g. brushed metal). In some cases, the BRDF of a visual feature leads unique signal behaviors based on the position between source, reflector, and detector. In some cases, these effects are used to apply specific reflection/absorption filters based on angular incidence, or are used for signal boosting by varying the source location. Furthermore, in some cases, the BRDF is employed in the spectral domain, where certain colors reflect more or less than others at given incident and reflection angles.

In the example shown in FIG. 5C, a computer takes as input: (a) data indicative of the intensity (and in some cases, color) of reflected light, (b) data indicative of the angle of reflectance of the reflected light (e.g., data regarding the position of a light source), and (c) data indicative of a BRDF. Taking this input, the computer calculates a position of a control feature, maps this position to a control signal, and then outputs the control signal.

In some implementations, multi-colored feature patterns simplify the distinction between different movable mechanical interfaces and the distinction between control or calibration features. In some implementations, scattering and absorption-specific pigments are used to differentiate visual feature types or their assigned roles. In some cases, optical polarization combined with pigments and optical properties also are used in order to distinguish between visual features.

A variety of optical patterns may be used for the visual features (e.g., control features or calibration features). In some cases, a circular dot is used (e.g., for displacement tracking of a mechanical component). In some cases, an elongated or line-like feature is used (e.g., for tracking rotation). In some cases, a calibration feature covers the control feature's travel range. For example, in some cases, such a calibration feature (which designates a specific area for feature detection), comprises a rectangle, a ring, or a ribbon that outlines a travel range of a control feature. In some cases, a checkerboard is used for calibration. In some cases, a barcode is used.

In illustrative implementations, a computer processes images of mechanical inputs of the hardware (including analyzing changes in the control canvas, mapping these changes to control signals, and outputting the control signals) at extremely short processing times. This very rapid processing is facilitated by using these optical patterns.

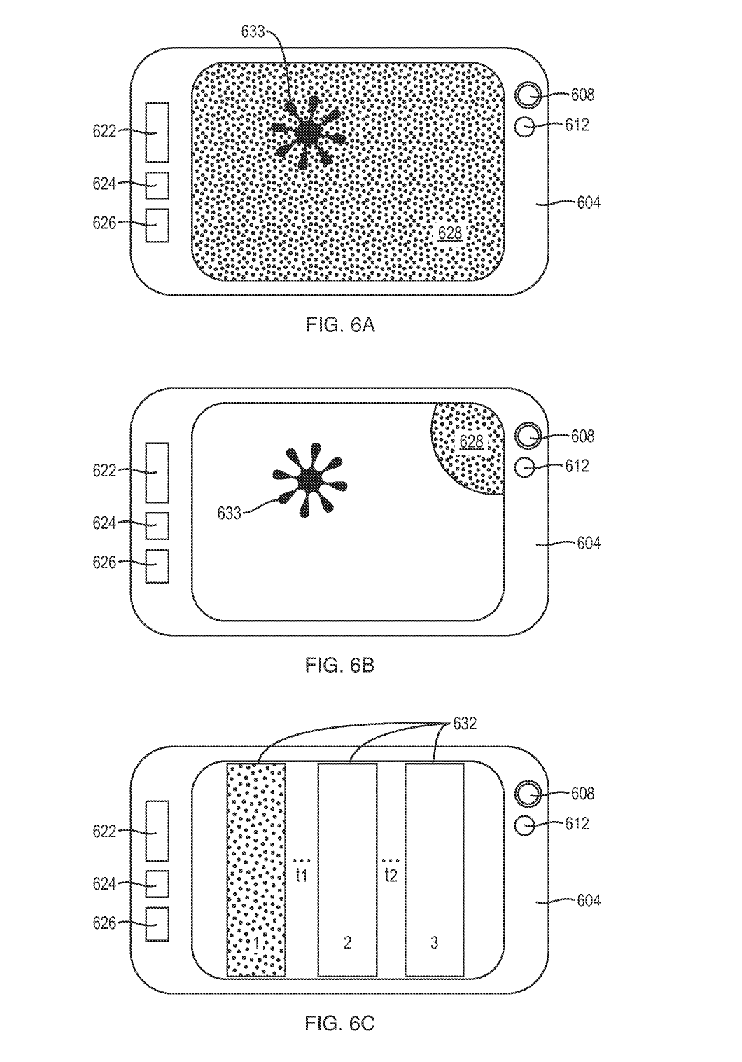

FIGS. 6A, 6B and 6C show three examples of light sources emitted by an MCD 604.

In FIG. 6A, the entire screen of the MCD 604 is used as a light source (indicated by black dots) 628. In FIG. 6B, a portion, but not all, of the screen of the MCD 604 is used as the light source (indicated by black dots) 628. In FIG. 6C, the region of the MCD screen that is used as a light source (indicated by black dots) 632 changes position over time. In FIGS. 6A, 6B, 6C, the light source 628, 632 illuminates control features and calibration features in the controller (not shown).

In FIG. 6A, a graphic feature 633 is displayed on the MCD screen, and thus is co-located with part of the light source 628.

In FIG. 6B, the graphic image 633 is displayed in a region of the MCD screen that is separate from the portion of the MCD screen used as a light source 628.

In FIGS. 6A, 6B and 6C, the MCD includes a computer 622, memory device 624, a wireless communication module 626, a camera 608 and LED flash 612.

In illustrative implementations, the MCD includes one or more point light sources and one or more spatial light sources, each of which are controlled by a computer onboard the MCD. For example, in some cases, a point light source onboard the MCD comprises a high intensity LED units used for flash photography. In some cases, a spatial light source onboard the MCD comprises a raster graphics display, liquid-crystal display (LCD), light-emitting diode (LED) display, organic-light-emitting diode (OLED) display, or electronic ink (E Ink) display. This invention is not limited to any particular type of light source. Any point or spatial light source that illuminates the control canvas may be used.

In exemplary embodiments of this invention, the MCD screen emits light to illuminate visual features of the handheld controller. The light is spatially uniform or has a spatial intensity gradient. In some cases (e.g., FIG. 6A), the entire available display area of the MCD screen is used for the light source and the displayed graphics are superimposed on the light source. In some cases, only a subsection of the display screen is used as a light source. This allows the system to decouple display graphics from the light source by separating both spatially. In some cases, the subsection is positioned so as to improve signal performance of the system by boosting the received signal from the control canvas. For example, in some cases (e.g. FIG. 6B), the light source is a corner area of the screen that is close to the camera and retroreflectors are used as control features in the canvas, causing the detected signal to be significantly enhanced allowing for a faster or more reliable update frequency due to lower required exposure time. Similarly, in some cases, concave parabolic mirrors or metallics redirect a portion of the light back to the camera, regardless of the light pattern. The narrower the reflection profile of the material on the canvas, the more precise the location or calibration algorithms become.

In illustrative implementations, the intensity of the light from the MCD is constant, time varying, or a mixture of both. In some cases, the update frequency of the optical link (between the MCD camera and visual features of the controller) is limited by the light detector's reading rate (i.e. frame rate). In some cases, for time varying implementations, the light from the MCD is periodically on/off-pulsed or alternated between selected intensity ranges. In some implementations, the light source and light detector are time-synchronized. Given the short distances involved and the speed of light, the time that it takes for light to travel from the MCD to the visual features and back is so short that it is treated as instantaneous, for computational purposes. With this instantaneous travel time, the beginning of every source pulse period marks the time when the light detector is triggered for signal acquisition.

In some cases, timing implementations are enhanced by using a light source that changes positions over time, as depicted in FIG. 6C. Selected display segments are periodically pulsed on/off (i.e. spatial pattern coded) such that the area of the screen that acts as the light source changes over time. For example, in FIG. 6C there are three partial display segments that are pulsed at a given rate and sequence. In many configurations (including those shown in FIGS. 6A and 6C), the area that acts as a light source and the displayed graphics are superimposed or co-located.

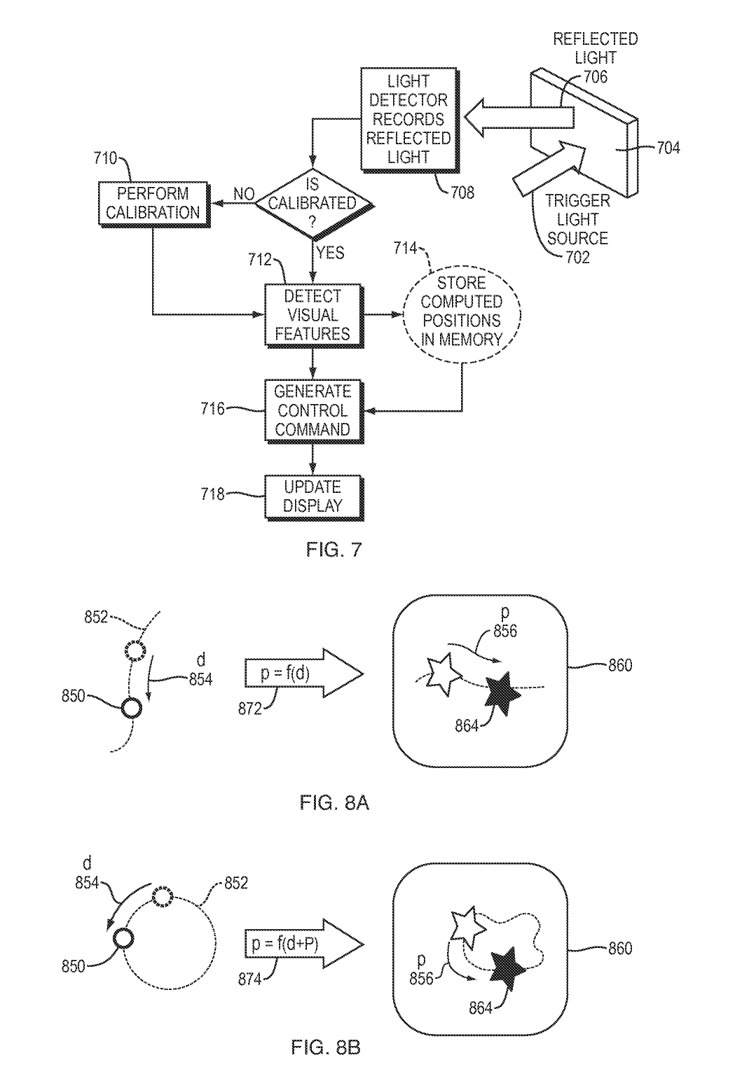

FIG. 7 is a conceptual diagram that shows steps in for a method for using a handheld controller to control operation of an MCD, in an exemplary implementation of this invention. In the example shown in FIG. 7, the method includes the following steps. Trigger the light source, according to a given set of parameters such as intensity or timing functions, such that light from the light source illuminates the control canvas 704 (Step 702). Use the light detector to record the reflected light 706 from the control canvas (Step 708). Use a computer onboard the MCD to check if a system calibration has been performed, and if not, perform calibration (Step 710). In step 710: (a) if calibration has not been performed before, a computer collects relevant system information, calibrates the system, and stores the calibration parameters in memory; and (b) if calibration has been performed, a computer loads calibration parameters from memory to access parameters found in previous cycles. Use a computer to detect visual features, by analyzing the recorded light data and extracting the positions of control features (Step 712). Store the computed positions of the visual features in memory (Step 714) for reference in subsequent program iterations. Generate a control command for each detected component by using the detected feature positions and comparing them to positional information gathered during previous program iterations, if available (Step 716). Update a graphical display, in accordance with the control command (Step 718). In some cases, the command signal triggers other events in the MCD to be executed immediately or at a future point in time. The method is then repeated by triggering the light source.

In illustrative implementations, system calibration is used to provide a stable and high quality control link between the controller and the MCD. Knowing the relative spatial positions of the visual features in an image frame and the allowed range of their movement paths is desirable for rapid processing. Calibration is desirable because the optical properties of an MCD camera vary between different MCD models, series, and makes. Among other things, differences in optical lenses, CCD chip size, chip light sensitivity, optical axis relative to the device, and camera location may cause large variations between the spatial location and size of features within images taken from the different MCDs.

In illustrative implementations of this invention, calibration is performed initially and during operation of the system. In many cases, calibration is performed on the program's first cycle to collect initial parameters of the system. However, in some use scenarios, aspects of the system change during runtime such as positional variance of the MCD with respect to the control canvas. In these scenarios, it is useful to trigger a calibration step on the next program cycle. In some cases, certain calibration steps are performed on every program cycle, while others are performed sparsely or only once.

In illustrative implementations, a camera is used as a light detector that provides the calibration or feature detector a new raster image on every new program cycle. The image contains visual data regarding the control canvas, from which the positional information of the various control and calibration features are extracted by a computer. For example, in some cases, a computer uses well established image processing algorithms to find calibration and control features and to record their positions with respect to the raster image coordinates. For example, in some cases: (a) a certain visual feature in the control canvas is known to be a round dot; (b) a computer onboard the MCD analyzes the image with a blob-detector algorithm to find the general location of the dot; and (c) the computer calculates the centroid of the pixels corresponding to the dot to achieve subpixel positional accuracy, thereby more accurately determining the center location of the dot with respect to the image coordinates.

FIG. 8A shows an example of using a control feature position in the control canvas 402 to calculate a position of a graphic feature that is displayed on a screen 860 of an MCD. In the example shown in FIG. 8A, a computer performs an algorithm that includes the following steps: (a) detect a control feature 850 in a camera image; (b) determine the control feature's position relative to a predefined path 852; (c) compare the position to one or more positions stored in memory, in order to calculate a path change d 854 (the path change d 854 being a change in position along path 852); (d) map the path change d 854 (of the control feature in the control canvas) to movement p 856 (movement p being a straight or curving motion of graphic feature 864 in the display 860); (e) store current control feature positions, graphics positions, path changes, and other relevant parameters are then stored in memory for use in subsequent iterations, and (f) output a control signal to update the graphic display of the MCD to display graphic feature 864 in a new position that is changed from its previous position by movement p. In this algorithm, a computer determines movement p by either using a mapping function p=f(d) 872 or by accessing a lookup table.

The function p=f(d) 872 or lookup table is predefined or is determined through one or more system calibration methods. In many implementations, a predetermined function p=f(d) is defined and then combined with a scaling factor c (e.g. pixels/millimeters) that is determined through system calibration. For example, as a user presses on an I/O and causes, by mechanical pressure, control features to move along a physical path in the control canvas, a camera detects the movement, and a computer onboard the MCD outputs control signals to cause a graphic image on the MCD display to move by a displacement that is scaled by c in distance. In many implementations: (a) the function p does not represent a one-to-one positional mapping from control canvas to MCD display; and (b) the function p instead skews the control feature path, rotate the path about a point, invert movement directions, or cause the graphics to move along an entirely different path characteristic than that of the control feature.

In some cases, the mapping function p=f(d) 872 is finite or periodic. A mechanical slider that moves along a fixed path causes the corresponding control feature to change its position by path change d 854 as illustrated in FIG. 8A. Similarly, a mechanically rotating interface in the canvas causes the corresponding control feature to move in a continuously looping path 852, as illustrated in FIG. 8B. In the latter example, the mapping of the control feature position to the displays graphics is determined via a periodic function p=f(d+P) 874, where P is the path length.

In some cases, the mapping function p=f(d) is applied to one or more graphic features. That is, in some cases, a single control feature controls the positions of multiple graphic features. Alternatively, in some cases, multiple control features drive their own mapping functions in an ensemble that controls the position of one or more graphics simultaneously. In some cases, a computer dynamically alters function p during system operation via system calibration.

In some cases, a computer recognizes errors in detection of control features (such as detecting control features that do not match a defined path or failing to recognize visual features), and then takes precautionary steps, such as determining whether (a) hardware (e.g., a visual feature) in the controller is broken, (b) the MCD is damaged, or (c) the connection between the MCD and the controller is damaged. In some cases, a computer also outputs control signals to cause an I/O device to notify a human user to take actions to correct the problem.

In many implementations, the exact path of control features is not known prior to system operation. Image calibration is performed to determine the locations and paths of control features. In some cases, image calibration removes distortions associated with the optical quality of the camera assembly and perspective deformations, and allows the interchangeability of MCD makes, series, and models within a single hardware attachment unit, or vice versa.

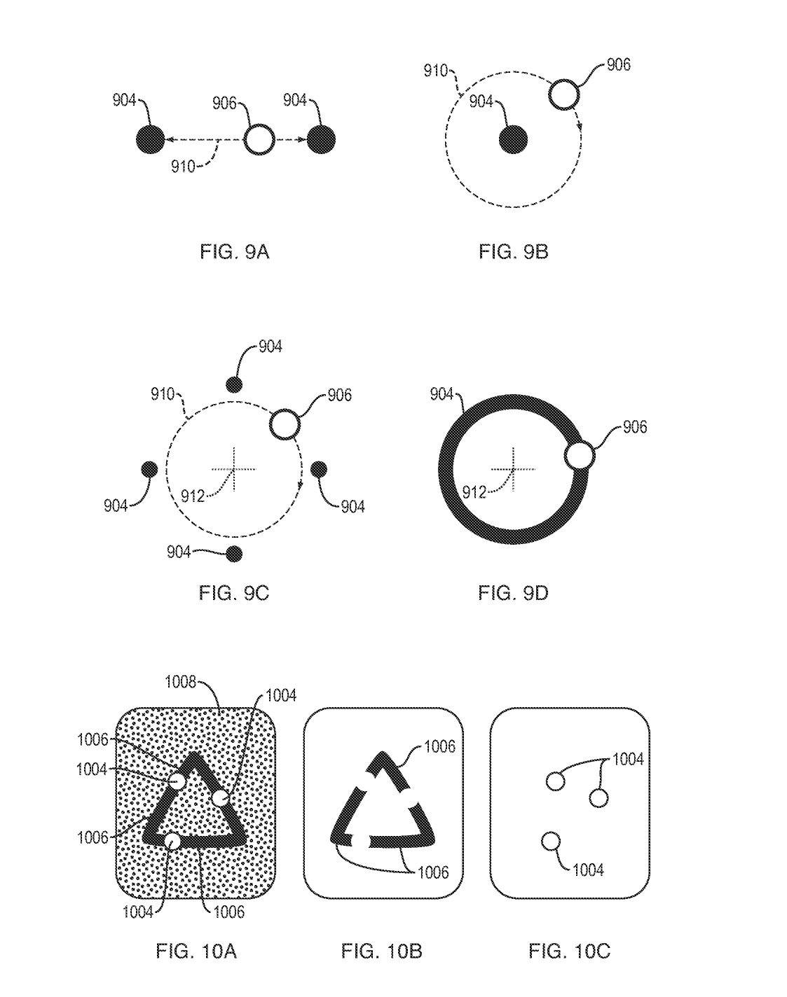

FIGS. 9A to 9D illustrate the use of calibration features 904 to determine the position and path 910 of control features 906, in illustrative implementations of this invention. In these examples, a computer determines the path of one or more control features by using calibration features as anchor points.

In some cases, a control feature moves along a finite path with given start and end points, as shown in FIG. 9A. Two calibration features 904 are positioned, one at each end of the path. A computer uses the two calibration features to determine distance and position of the path 910 during calibration.

In some cases, calibration features are positioned at a known offset from a control feature path, as shown in FIGS. 9B and 9C.

In some cases, one or more control features are placed on top of calibration features. In some cases, one or more calibration features trace the entire path of a control feature (as shown in FIG. 9D) rather than indicating merely the start or end points.

In some cases, an elongated calibration feature is positioned such that the calibration feature is offset from and parallel to an elongated path of a control feature. In some cases, the topological range (i.e. feature elevation relative to the camera's perpendicular plane) is determined by positioning calibration features at the apex and base of a control feature's elevation range (elevation with respect to the control canvas plane). In some cases, features that have no distinct path, but rather, are predictably located within a given area or “zone” are surrounded by a box-like calibration feature that indicates the allowed feature location area. In some cases, a calibration feature is used that indicates an area that should be free of control features.

In some cases: (a) the control feature travels in periodic movements; (b) the control feature path circumscribes a region; and (c) one or more calibration features demark a center point 912 at the center of the region. In some implementations, the center point is indicated directly by placing a control feature at the center location of the rotating mechanical interface, as is illustrated in FIG. 9B. In some cases: (a) a computer performs an algorithm that accurately determines a center point even when a relatively large control feature is used; and (b) the algorithm includes detecting the control feature and calculating the centroid based on surrounding pixel intensities in the image.

In some implementations, a set of calibration features are placed around a component in the controller, in order to indicate the position of one or more points in the component, as shown in FIG. 9C. This approach is advantageous is some cases, such as where it would be difficult to place a calibration feature at the center of a component (e.g. it would be difficult to place a calibration feature at the center of a viewing port 450 or of optical lenses). In some cases in which multiple calibration features are placed around a component, a computer determines the center point of the component by using positional averaging or by using geometric methods such as a bisector-intersection calculation. In some cases in which calibration features surround the allowed path of a control feature, a computer calculates the radius of the path.

In some implementations: (a) a calibration feature spans the entire control feature path, as shown in FIG. 9D; and (b) a computer uses the calibration feature to determine exact path placement and also determine path parameters such as the path radius or ellipticity.

The examples in FIGS. 9A, 9B, 9C and 9D show linear or circular path implementations. However, this invention is not limited to linear or circular paths. For example, in some cases, calibration features are in any shape or form (e.g. triangular, oval, or more complex paths).

In some implementations, the control features themselves are used to calculate the control feature path and position. This is advantageous, for example, where: (a) no calibration features are available, or (b) a given mechanical interface does not support calibration features. In some cases (in which the control features are used to calculate the control feature path), the control features are moved into all their possible states while tracing the positions and storing intermediate positions into memory. This calibration method is done in advance by saving the path of each control feature, or during the system operation by using a “learning” algorithm while the user operates the system.

Alternatively, calibration is performed without using calibration features by taking a series of images in succession while the user operates the hardware attachment such that all possible positions, paths, and areas of the given set of control features are reached. A computer combines pixel values of each image frame using a non-maximum-suppression technique and outputs a composite image of the feature position space. This composite image maps out areas in which features are expected to be present and areas in which features are expected to be absent during normal operation.

In a separate implementation, a computer uses calibration features to determine positions of the control canvas, light source, light detector, and display unit relative to each other (e.g. perpendicular distance between control canvas plane and display unit plane in millimeters). In some cases, a computer calculates these positions by detecting calibration features with known absolute displacements, and then combining this information with known MCD parameters such as the distance between the light detector unit and the display unit.

In some implementations, color segmentation is used to aid system calibration and to improve the signal-to-noise ratio (SNR) of the content within image frames. Color segmentation is implemented by either the light source, the light detector, or both. For example, in some cases, the color range of the light source is selected, such that the SNR of a given control feature is enhanced to spatially filter out the calibration features and background information from the detector signal. In some cases, the color range of light detector data is controlled through color channel filtering.

In some implementations, different areas of the control canvas have different spectral responses to light. For example, for a first color of light, a first region of the control canvas may reflect more light than a second region of the control canvas does, and for a second color, the first region of the control canvas may reflect less light than the second region does.

FIGS. 10A, 10B, 10C show steps in a method for determining a control feature path in a noisy image, by varying the color of a light source, in an illustrative implementation of this invention. In the example in FIG. 10A, a light source emits a broad visible spectra (e.g. white light) resulting in a response from all elements in the control canvas. The light source is then switched to a specific color, e.g. blue monochrome, which visually enhances calibration features 1006 that reflect predominantly blue light, while simultaneously suppressing control features 1004 and background elements 1008 that absorb blue light. The resulting filtered image is shown in FIG. 10B. A computer takes the filtered image as input, and determines the allowed path of the control feature. Then, the light source is switched to a different color, e.g. yellow monochrome, which singles out control features 1004 that reflect predominantly yellow light, while simultaneously suppressing calibration features 1006 and background elements 1008 that absorb yellow light. The resulting filtered image is shown in FIG. 10C.

Similarly, in some cases, the data collected by the light detector is color segmented to achieve visual feature separation. For example, in some cases, a CCD camera in the MCD operates using three distinct color channels (i.e. RGB: red, green, blue), and features that appear in one color channel are segmented from features appearing in one or both of the other channels. In some cases, RGB color channel data is reformulated to other color spaces, such as YUV, CMYK, or L*ab, thereby providing additional options in channel segmentation. For example, in some cases, using the red chroma channel (Cr in YUV color space) significantly enhances features with a red tone, while strongly suppressing features with a blue tone. Color segmentation methods are advantageous in low-light environments with limited light detector sensitivity.

In some implementations, other noise reduction techniques are used to enhance the SNR of control features during system operation.

For example, in some cases: A series of “ground truth” images are captured during system calibration. The ground truth images can be subtracted from frames captured during system operation, which results in composite images that are void of background image content. In some cases, the active light source is turned off when acquiring the ground truth images. This causes the information in the captured frames to be effectively a snapshot of undesirable image noise content under ambient light. A computer treats the noise image as a ground truth and subtracts the noise image from subsequent image frames during system operation. In some cases, noise reduction techniques are used prior to the main system operation time, as a calibration step, or triggered any time during the system operation. For example, in some use scenarios, an optical link between controller and MCD is determined to be unsatisfactory, and a new ground truth snapshot sequence is triggered by briefly turning the light source off, capturing an image frame, and then turning the light source back on.

In some cases: (a) reflection/absorption spectra of visual features are not known in advance; and (b) a color sweep is performed during system calibration by varying the color of the light source. A first color is emitted from the light source, and the response from each visual feature is measured by the light detector. This is repeated for a variety of different colors. A computer compares the color response measurements from each visual feature, and selects the color/detector-sensitivity combinations that favor optimal feature segmentation. In some cases, a computer dynamically adjusts the color range of the light source during system operation, in order to optimize the control feature response in each image frame versus the light detector's sensitivity.

Alternatively, in some cases: (a) reflection/absorption spectra of visual features are not known in advance; and (b) a color sweep is performed during system calibration by using a constant light source color, but computationally altering the color response of the light detector by sweeping through color channels, color spaces, and hue levels in each image frame.

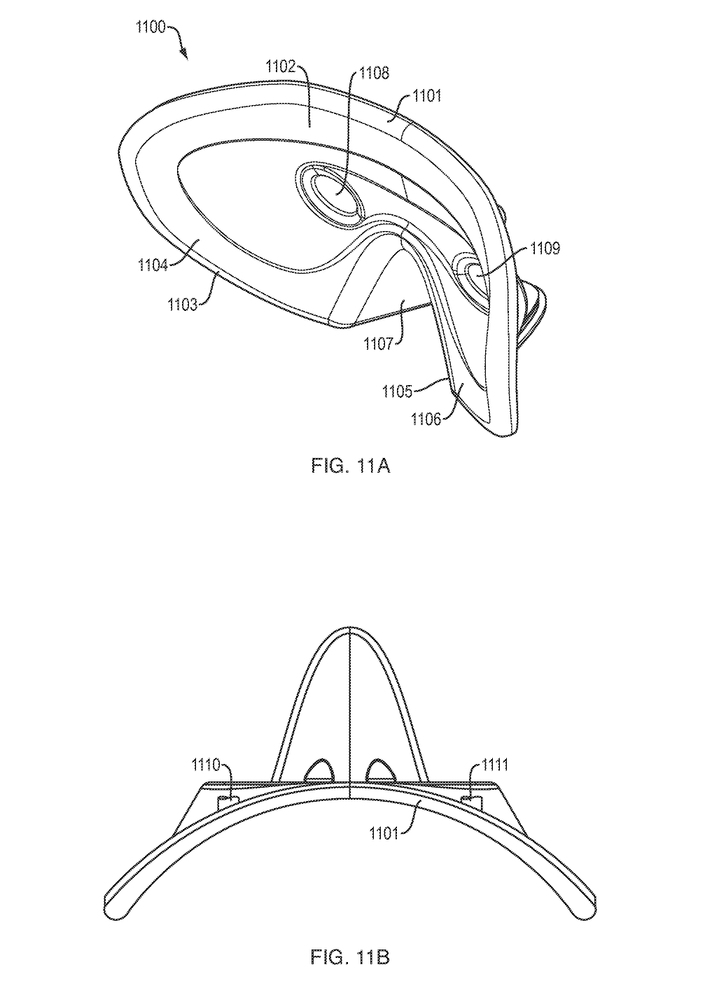

FIGS. 11A, 11B, 11C and 11D are four views of a face-fitting portion 1100 of the controller. The face-fitting portion is configured to be pressed against at least the forehead and cheeks of human user. FIG. 11A is a perspective view; FIG. 11B is a top view; FIG. 11C is a back view (seen from the vantage point of the human user's face); and FIG. 11D is a side view. Face-fitting portion 1100 of the controller forms a surface that includes multiple curved or planar regions. Regions 1101, 1102 are configured to be pressed against (and to fit snugly against, and to conform to the shape of) the forehead of a human, either at or above the brow ridges of the human. Regions 1103, 1104 are configured to be pressed against (and to fit snugly against, and to conform to the shape of) a cheek of a human. Regions 1105, 1106 are configured to be pressed against (and to fit snugly against, and to conform to the shape of) another cheek of the human. Region 1107 is configured to be pressed against (and to fit snugly against, and to conform to the shape of) the nose of the human. Eyeholes 1108 and 1109 are holes through which a human user looks, when portion 1100 is pressed against the face of the user. Structural posts (e.g., 1110, 1111, 1112) connect the face-fitting portion 1100 to the remainder of the controller. In FIG. 11D, a portion of the main body 1114 of the controller is indicated by dashed lines.

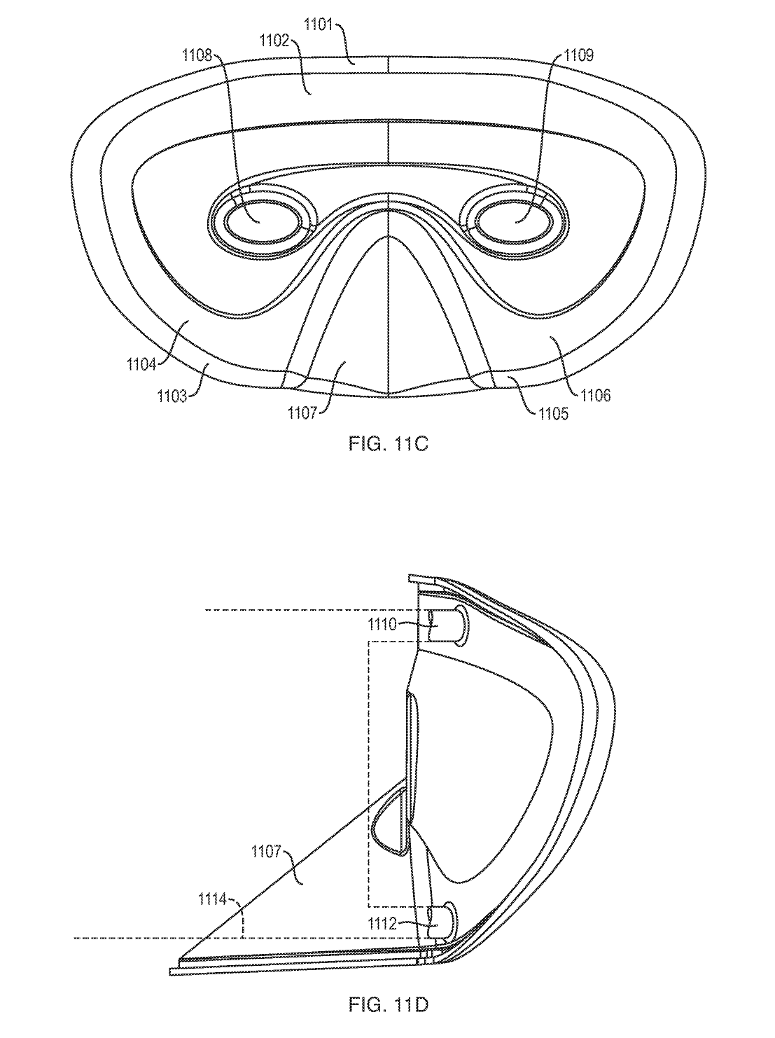

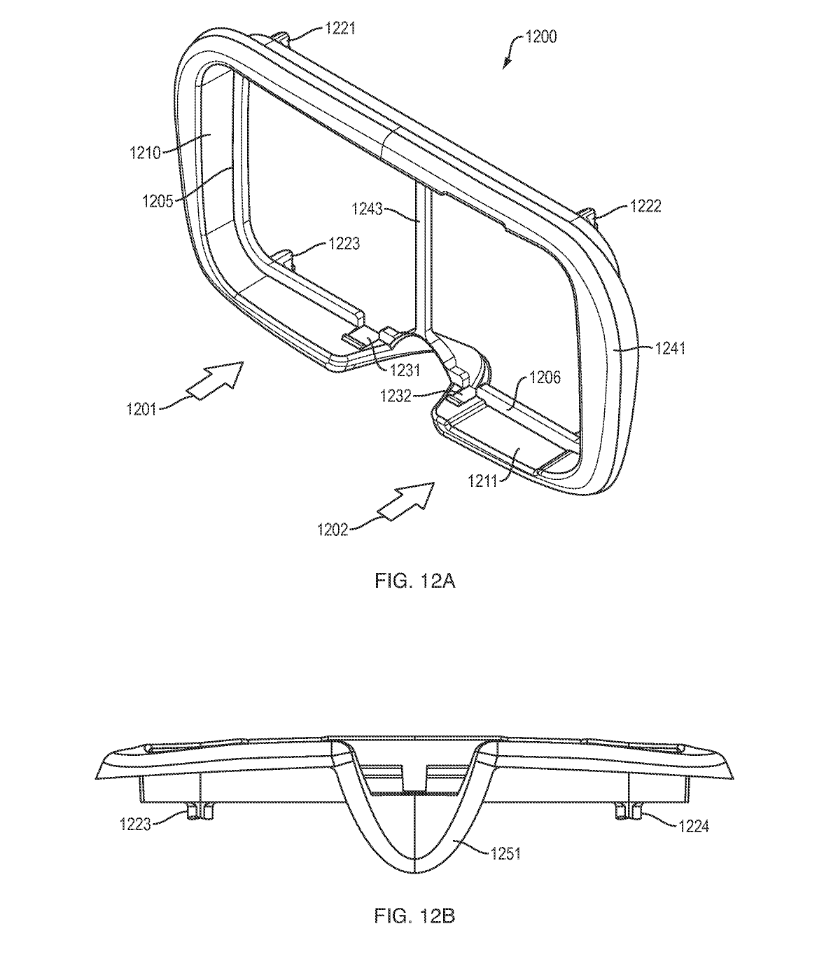

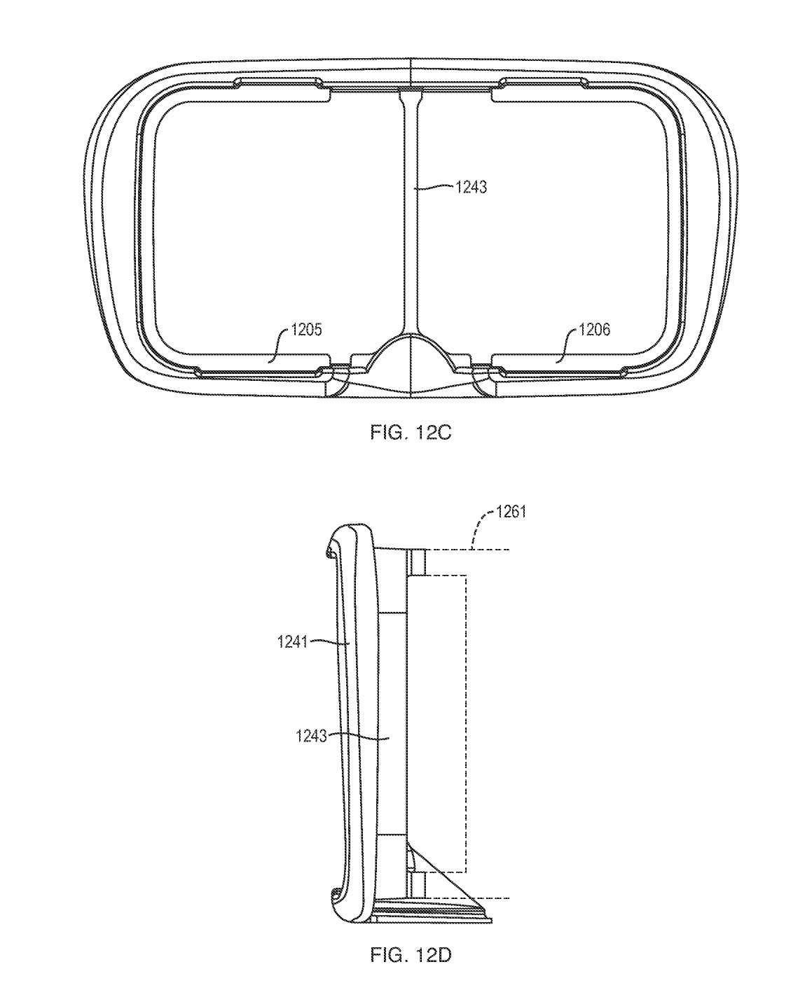

FIGS. 12A, 12B, 12C and 12D are four views of an attachment mechanism 1200 for attaching the controller to a mobile computing device (MCD), such that the controller is easily attached to and easily released from the MCD. FIG. 12A is a perspective view; FIG. 12B is a bottom view; FIG. 12C is a back view; and FIG. 12D is a side view. An opening in the attachment mechanism is surrounded by inner walls (e.g., wall 1210). The MCD is inserted into this opening in an insertion direction indicated by arrows 1201, 1202. The movement of MCD in the insertion direction is restrained by lips 1205, 1206. Tabs 1231, 1232 are flexible and press against the MCD when the MCD is touching lips 1205, 1206, tending to restrain movement of the MCD. The MCD is easily removed (released) from the attachment mechanism by pulling the MCD in a direction opposite to the insertion direction. A gentle pull on the MCD overcomes friction caused by the pressure exerted by tabs 1231, 1232 against the MCD. The indentation at 1211 creates a space such that user interfaces of the MCD do not press against the inner walls of the attachment mechanism when the MCD is inserted or removed from the attachment mechanism. This allows the MCD to be inserted and removed without inadvertently actuating these MCD user interfaces. The walls of the opening have an exterior surface, including region 1241. A support post 1243 connects two sides of the opening of the attachment mechanism, but is positioned such that it does not block the insertion and removal of the MCD. Region 1251 exposes part of the MCD to allow easier insertion of the MCD into the controller, or to allow easier removal of the MCD from the controller. For example, to remove the MCD, a user presses a thumb or other finger into the opening created by region 1251, presses the thumb or other finger against the MCD, and applies force to the MCD. Structural posts (including 1221, 1222, 1223, 1224) connect the attachment mechanism 1200 to the remainder of the controller. In FIG. 12D, a portion of the main body of 1261 of the controller is indicated by dashed lines.

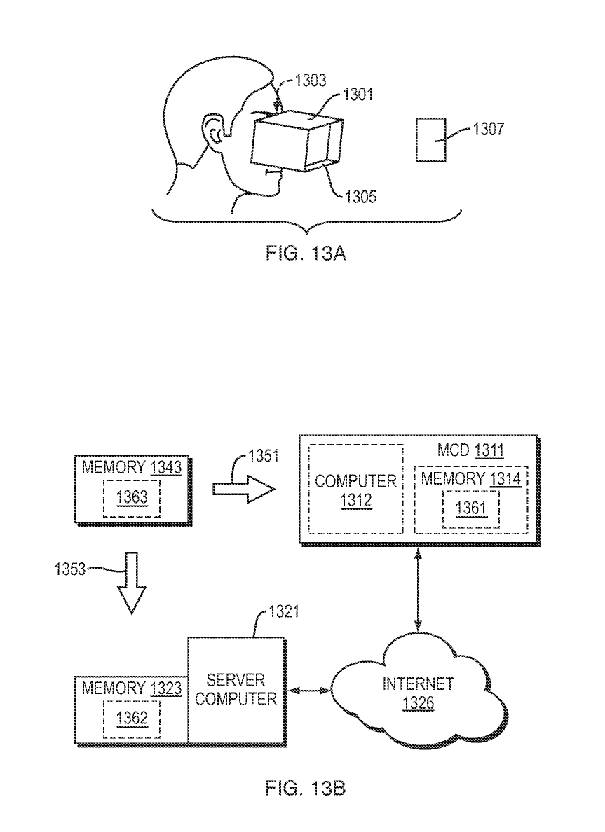

FIG. 13A shows an example of a system comprising a machine-readable medium and a controller. In the example shown in FIG. 13A, a handheld controller 1301 includes a surface 1303 that is configured to be pressed against the forehead and cheeks of a human user, and an attachment mechanism 1305 for attaching the controller to, and allowing the release of the controller from, an MCD. A machine-readable medium 1307 has program instructions encoded therein for a computer 1312 (i) to generate control signals that cause a camera onboard the mobile computing device to capture images indicative of the movement; and (ii) to process the images to recognize the movement and, based on data indicative of the movement, to generate control signals to control at least, at least in part, operation of the mobile computing device. Alternatively or in addition, the program instructions encoded on the machine-readable medium comprise instructions for a computer to perform any control task, calculation, computation, program, algorithm, computer function or computer task described or implied herein.

FIG. 13B shows three examples of locations for a machine-readable medium (e.g., 1361, 1362, 1363) that stores the encoded program instructions, in illustrative implementations of this invention.

The first example is onboard an MCD. In FIG. 13B, memory device 1314 and computer 1312 are onboard the MCD 1311. In some cases, memory device 1314 is an internal memory unit in computer 1312 or an auxiliary or external memory unit for computer 1312. A handheld controller (e.g., 202) is attachable to the MCD 1311. Machine-readable medium 1361 comprises a portion of a memory device 1314. Computer 1312 executes the encoded program instructions that are described in the discussion of FIG. 13A, above.

The second example is in memory for a server computer. In FIG. 13B, machine-readable medium 1362 comprises a portion of a memory device 1323. In some cases, memory device 1323 is an internal memory unit in a server computer 1321 or an auxiliary or external memory unit for server computer 1321. Server computer 1321 is connected to the Internet 1326. A copy of the encoded program instructions that is stored in the machine-readable medium 1362 is downloaded, via the server computer 1339 and Internet 1326, and is installed as an app in MCD 1311 and stored in memory device 1314 onboard the MCD 1311. After the app is installed on the MCD, computer 1312 onboard the MCD executes the encoded program instructions. In some cases, multiple users, each of whom have a handheld controller, access the server computer 1321 via the Internet 1326, in order to the download a copy of the instructions and to install the copy as an app on an MCD.

The third example is in a master copy. In FIG. 13B, a machine-readable medium 1363 comprises a portion of a memory device 1343, and stores a master copy of the encoded program instructions. In some cases, during manufacture of an MCD, the encoded program instructions of the master copy are copied 1351 from memory device 1343 and the copied instructions are stored in a memory device 1314 onboard the MCD. In some cases, the encoded program instructions of the master copy are copied 1353 from memory 1343 and the copied program instructions are stored in memory device 1323 for server computer 1321.

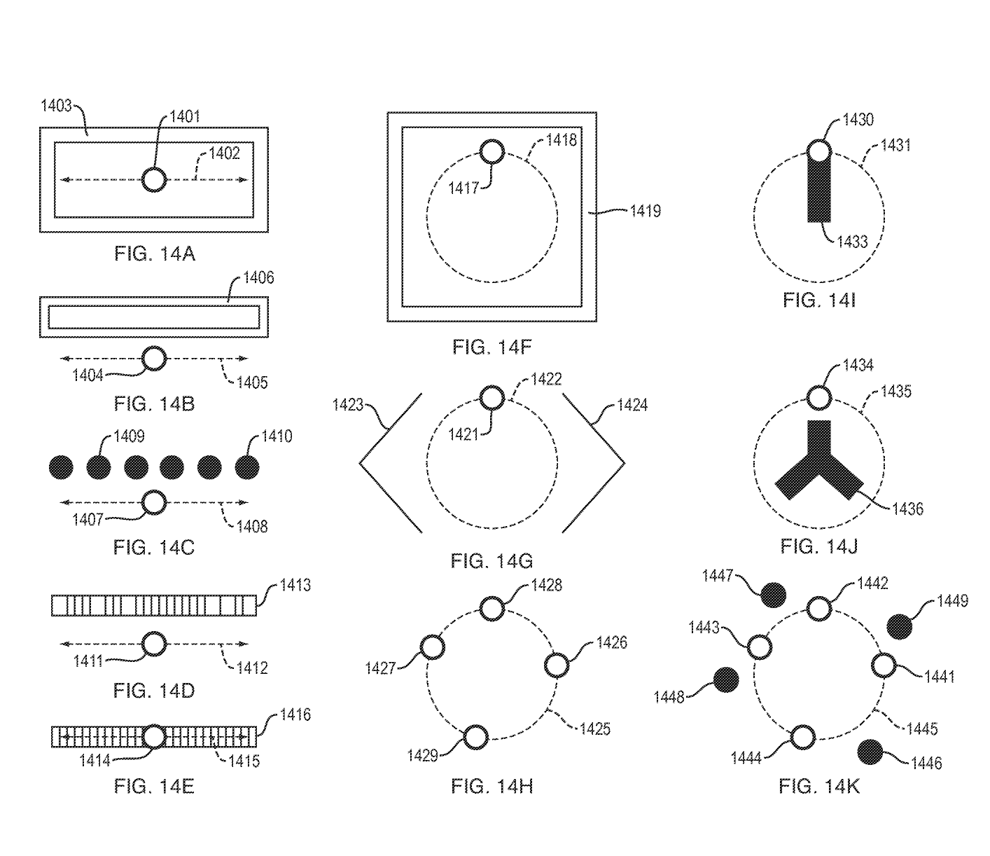

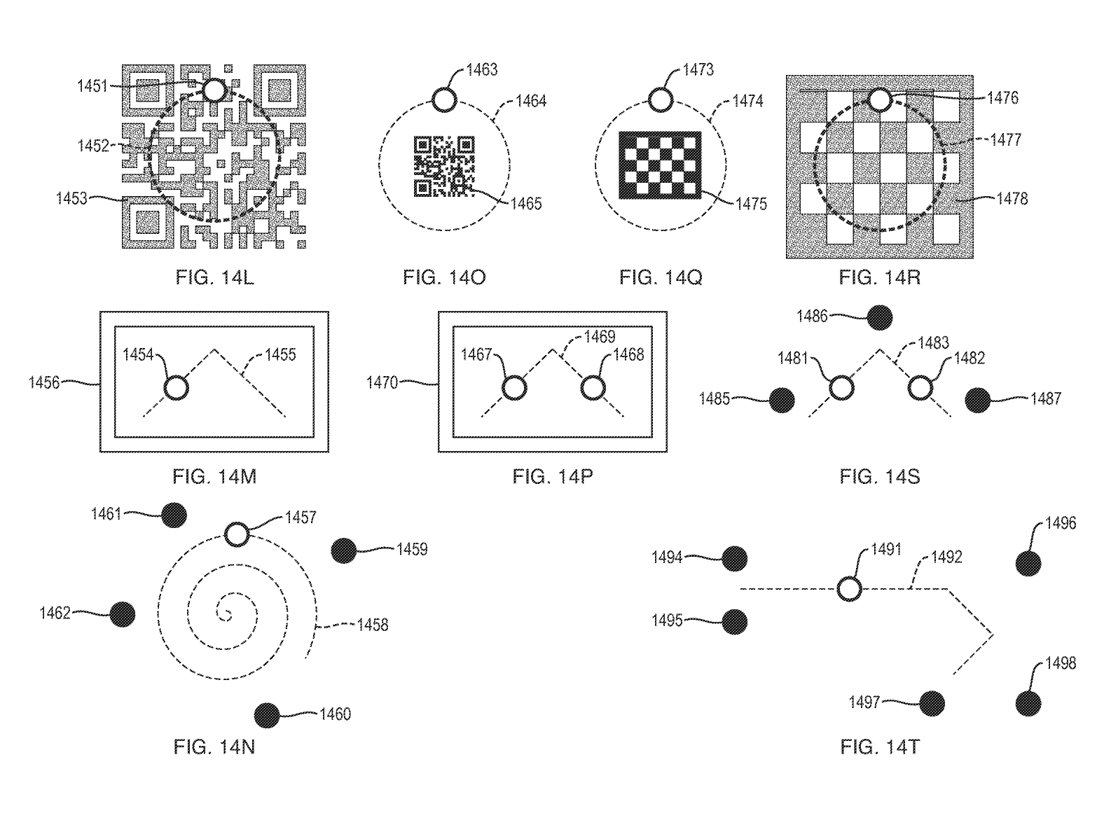

FIGS. 14A, 14B, 14C, 14D, 14E, 14F, 14G, 14H, 141, 14J, 14K, 14L, 14M, 14N, 14O, 14P, 14Q, 14R, 14S, and 14T each show a different example of one or more calibration features that are indicative of a path of a control feature. In each of these examples, the path comprises the set of all locations to which a control feature (or set of control features) is allowed to move relative to the controller. In each of these examples (in FIGS. 14A-14T), a computer analyzes camera images to determine positions of control features, calibration features, to determine the path of the control features, and to determine path changes.

In FIG. 14A, calibration feature 1403 indicates the position of a straight path 1402 for a control feature 1401. The path 1402 is located inside the calibration feature 1403. Control feature 1401 moves along path 1402.

In FIG. 14B, calibration feature 1406 indicates the position of a straight path 1405 for a control feature 1404. The path 1405 is located outside of, and parallel to the longitudinal axis of, control feature 1404. Control feature 1404 moves along path 1405.

In FIG. 14C, a set of calibration features (including 1409, 1410) indicates the position of path 1408 for a control feature 1407. Path 1408 is offset from the set of calibration features. Control feature 1407 moves along path 1408.

In FIG. 14D, calibration feature 1413 is a barcode pattern. The position of calibration feature 1413 indicates the position of path 1412 for control feature 1411. Path 1412 is offset from calibration feature 1413. Control feature 1411 moves along path 1412.

In FIG. 14E, calibration feature 1416 indicates the position of path 1415 for a control feature 1414. Control feature 1414 moves along path 1415.

In FIG. 14F, calibration feature 1419 indicates the position of circular path 1418 for a control feature 1417. Control feature 1417 moves along path 1418.

In FIG. 14G, calibration features 1423, 1424 indicate the position of circular path 1422 for a control feature 1421. Control feature 1421 moves along path 1422.

In FIG. 14H, there are no calibration features. During calibration, control features 1426, 1427, 1428, and 1429 are moved to all possible positions along a path 1425. The different positions are recorded.

In FIG. 14I, calibration feature 1433 indicates the position of path 1431 for control feature 1430. Control feature 1430 moves along path 1431.

In FIG. 14J, calibration feature 1436 is Y-shaped. Calibration feature 1436 indicates the position of circular path 1435 for control feature 1434. Control feature 1434 moves along path 1435.

In FIG. 14K, calibration features 1446, 1447, 1448, 1449 indicate the position of circular path 1445 for control features 1441, 1442, 1443, 1444. Control features 1441, 1442, 1443, 1444 move along path 1445.

In FIG. 14L, calibration feature 1453 comprises a pattern of dark and bright areas. Calibration feature 1453 indicates the position of path 1452 for control feature 1451. Control feature 1451 moves along path 1452.

In FIG. 14M, calibration feature 1456 indicates the position of bent path 1455 for control feature 1454. Control feature 1454 moves along path 1455.

In FIG. 14N, calibration features 1459, 1460, 1461, 1462 indicate the position of spiral path 1458 for control feature 1457. Control feature 1457 moves along path 1458.

In FIG. 14O, calibration feature 1465 is a pattern of dark and light areas. Calibration feature 1465 is located inside of, and indicates the position of, circular path 1464 for control feature 1463. Control feature 1463 moves along path 1464.

In FIG. 14P, calibration feature 1470 indicates the position of bent path 1469 for control features 1467, 1468. Control features 1467, 1468 move along path 1469.

In FIG. 14Q, calibration feature 1475 is a checkerboard pattern that is located inside of, and that indicates the position of, circular path 1474 for control feature 1473. Control feature 1473 moves along path 1474.

In FIG. 14R, calibration feature 1478 is a checkboard pattern that indicates the position of circular path 1477 for control feature 1476. Control feature 1476 moves along path 1477.

In FIG. 14S, calibration features 1485, 1486, 1487 indicate the position of bent path 1483 for control features 1481, 1482. Control features 1481, 1482 move along bent path 1483.

In FIG. 14T, calibration features 1494, 1495, 1496, 1497, 1498 indicate the position of irregularly shaped path 1492 for control feature 1491. Control feature 1491 moves along path 1492.

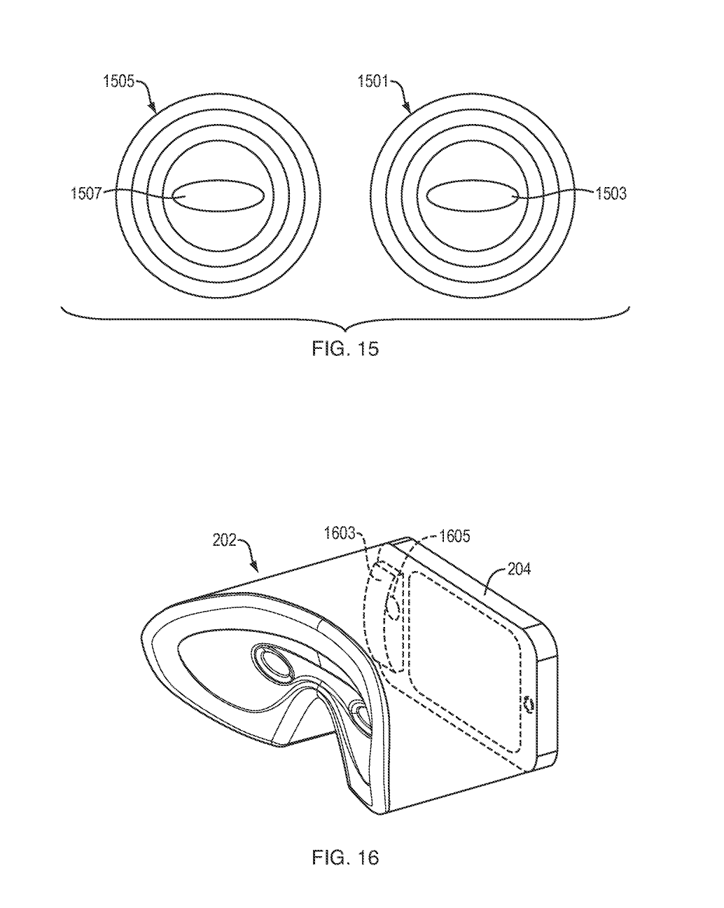

FIG. 15 shows an example of concentric rings around eyeports. A first set of concentric rings 1501 surrounds eyeport 1503; and a second set of concentric rings 1505 surrounds eyeport 1507. In some cases, the rings 1501, 1505 comprise active light sources, such as LEDs arranged in a circular shape. In some cases, the rings 1501, 1505 comprise passive light sources, such as reflective surfaces that reflect light emitted by the MCD. In some cases, the rings 1501, 1503 are used for corneal topography, as described below.

In some implementations of this invention, relay optics increase, decrease or shift a camera's field of view, and thereby (a) increase spatial resolution and (b) center the control components in a captured image. The increased spatial resolution facilitates optical tracking of visual features (e.g., 420) of moving control components (e.g., 406, 415, 419, 423) and increases the range (depth) of such optical tracking.

FIG. 16 shows an example of relay optics in a controller 202, in an illustrative implementation of this invention. In FIG. 16, the relay optics comprise a refractive optical element 1603 that is positioned over a camera 1605 in an MCD 204, when the controller 202 and MCD are attached to each other. The refractive optical element refracts light, such that the control canvas 402 is centered in images captured by the camera 1605. In some cases, the refractive optical element 1603 comprises a wedge with a variable radius of curvature. For example, in some cases, the wedge comprises an elongated slab wedge with trimmed sides. This wedge refracts light, thereby shifting the image captured by camera 1605, such that all of the control components of the control canvas are visible to the camera 1605. In FIG. 16, the refractive optical element 1603 is in the shape of a wedge. However, refractive optical element 1603 may have any shape. For example, in some cases, refractive optical element 1603 comprises a wedge, a prism, a plano-convex lens, a plano-concave lens, a convex lens, or a concave lens.

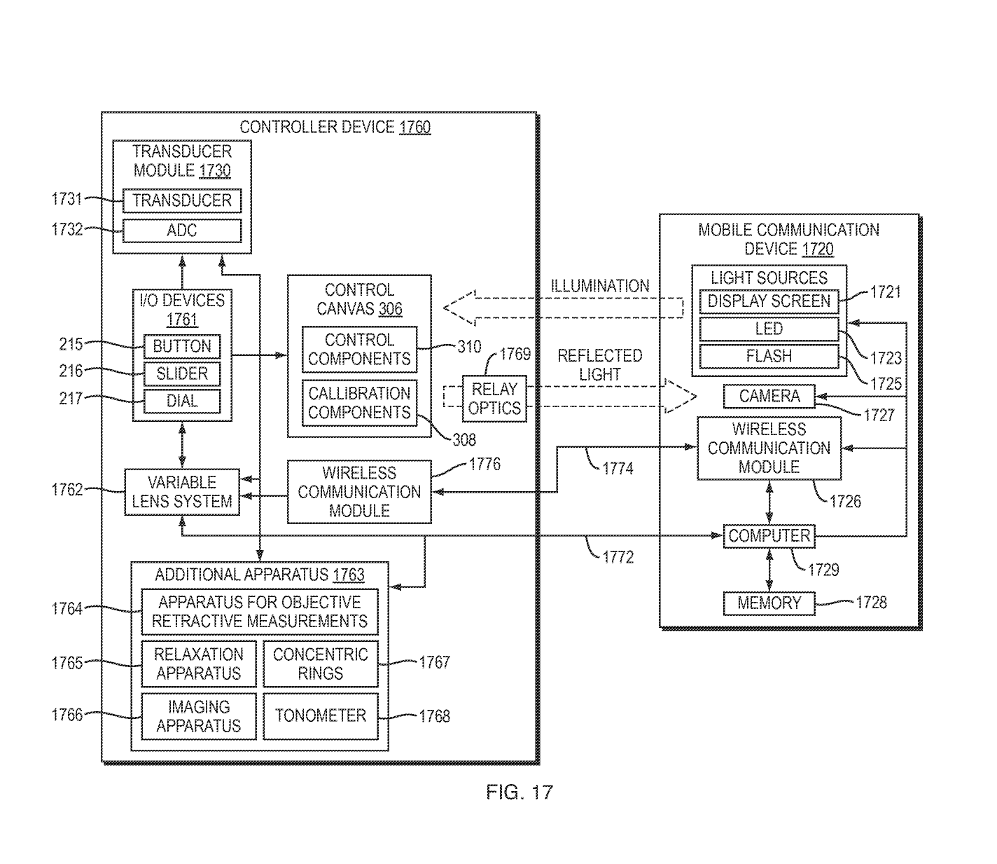

FIG. 17 is a block diagram of a controller device 1760 and MCD 1720, in an illustrative implementation of this invention. The controller device 1760 is releasably attached to MCD 1720.

In the example shown in FIG. 17, the controller device 1760 is either handheld (such that it can be held up to a user's eyes) or is worn on the user's head or otherwise head-mounted. In FIG. 17, the controller device 1760 includes a control canvas 306 (including calibration components 308 and control components 310), I/O devices 1761, and one or more of the following: (a) a variable lens system (VLS) 1762; (b) additional apparatus 1763; (c) relay optics 1769; (e) wireless communication module 1776; and (f) transducer module 1730. The additional apparatus 1763 includes one or more of the following: (a) apparatus for objective refractive measurements 1764, relaxation apparatus 1765, imaging apparatus 1766, concentric rings 1767 and tonometer 1768.

In the example shown in FIG. 17, the controller device 1760 includes a control canvas 306. The control canvas 306 comprises calibration components 308 and control components 310. The control canvas, including calibration components and control components and affixed visual elements, function as described elsewhere in this document.

The controller device 1760 includes I/O devices 1761, such as a dial 217, button 215, or slider 216. A human user presses against, or otherwise applies force to, the I/O devices 1761, in order to mechanically move the I/O devices 1761. The movement of the I/O devices 1761 is, in turn, mechanically transferred to control components 310, causing the control components 310 to move also.

The movement of the control components 310 is used to control operation of the MCD 1720 or apparatus onboard the controller 1760, as follows: One or more light sources onboard the MCD 1720 (e.g. a display screen 1721, LED 1723 or flash 1725) illuminate the moving control components 310. A camera 1727 onboard the MCD 1720 captures images of the moving control components 310 and of visual features attached to the moving control components 310. One or more computers 1729 onboard the MCD 1720 process the images and output control signals. In some cases, the control signals control operation of the MCD 1720, such as by controlling a visual display on a screen 1721 of the MCD 1720. In some cases, the control signals are sent to the controller device 1760 via a wired communication link 1772 or via a wireless communication link 1774. The wireless communication link 1774 is between wireless communication module 1726 (which is onboard the MCD 1720) and wireless communication module 1776 (which is onboard the controller device 1760). The control signals control operation of one or more devices onboard the controller device 1760, such as (a) a variable lens system 1762, apparatus for objective refractive measurements 1764, relaxation apparatus 1765, imaging apparatus 1766, concentric rings 1767 or tonometer 1768.

Alternatively or in addition, in some cases, at least some of the I/O devices 1761 are operatively connected to a transducer module 1730 onboard the controller device 1760. The transducer module 1730 converts mechanical motion into electrical energy. For example, in some cases, the mechanical motion is imparted by a human user manipulating at least some of the I/O devices 1761. The transducer module 1730 includes: (a) a transducer 1731 for transforming mechanical movement into analog electrical current or voltage; and (b) an ADC (analog to digital converter) 1732 for converting the analog electrical current or voltage into digital data. The digital data in turn controls the operation of devices onboard the controller device 1760, such as (a) a variable lens system 1762, apparatus for objective refractive measurements 1764, relaxation apparatus 1765, imaging apparatus 1766, concentric rings 1767 or tonometer 1768.

The controller device 1760 includes a variable lens system (VLS) 1762. One or more refractive attributes (e.g., spherical power, cylindrical power, cylindrical axis, prism or base) of the VLS 1762 are adjustable. The user holds the device 1760 up to his or her eyes, and looks through the device 1760 (including through the VLS 1762) at screen 1721 of MCD 1720. Iterative vision tests are performed, in which refractive properties of the VLS are changed from iteration to iteration. I/O devices 1761 onboard the controller device 1760 receive input from the user regarding which VLS setting results in clearer vision. For example, in some use scenarios, if spherical power is being optimized during a particular step of the testing procedure, the user inputs feedback regarding whether a test image appears clearer with the current VLS setting (a changed spherical power) than with the last VLS setting (a prior spherical power).

In the example shown in FIG. 17, a computer (e.g., computer 1729 onboard the MCD 1720) analyzes data gathered in these iterative vision tests, and calculates a refractive assessment. The refractive assessment specifies one or more refractive attributes (e.g., spherical power, cylindrical power, cylindrical axis, prism or base) for eyeglasses or contact lenses that would correct refractive aberrations of the user's right and left eyes, respectively. The refractive assessment is outputted, in human perceptible form, via one or more I/O devices (e.g., via screen 1721 of MCD 1720).

During the iterative vision testing, a mobile computing device (MCD) 1720 is attached to the front of the controller device 1760 (i.e., to a side of the device 1760 opposite the user's eyes). During the test, the scene a user sees (when looking through the controller device 1760) is an image displayed on a screen 1761 of the MCD 1720. For example, in some cases, the MCD 1720 comprises a smartphone or cell phone, and the user views all or portions of the phone's display screen when looking through the controller device 1760.

After the MCD 1720 is attached to the front of the controller device 1760, the user looks through the controller device 1760. Specifically, the user holds a viewport or eyeports of the controller device 1760 at eye level, and looks through the controller device 1760 to see the MCD screen 1721. The user sees light that travels through the controller device 1760: light travels from the MCD screen, then through the variable lens system (1762) of the controller device 1760, then through a viewport or eyeholes of the controller device 1760, and then to the eyes. The MCD 1720 is attached on one side of the device 1760; the viewport or eyeholes are on an opposite side of the device 1760.

During at least part of the iterative vision test, the MCD screen displays one or more visual patterns that are used in the test.

In illustrative implementations, the user gives feedback regarding which setting of the variable lens system (VLS) 1762 produces the clearest vision for the user. For example, in some use scenarios: (a) in a first trial, a VLS refractive attribute (e.g., spherical power, cylindrical power, cylindrical axis, prism or base) is set to a first value while the user looks through the controller device 1760 at a test image displayed on the MCD screen; (b) in a second trial, the VLS refractive attribute is set to a second value while the user looks through the controller device 1760 at the same test image on the MCD screen; and (c) an I/O device 1761 accepts input from the user regarding whether the image in the second trial looks clearer or less clear than in the first trial. The format of the input may vary. For example, in some cases, the user simply indicates which image he or she prefers, and this input regarding preference is a proxy for which image appears clearer to the user.

The VLS 1762 comprises one or more lenses and, in some cases, one or more actuators. One or more refractive attributes (e.g., spherical power, cylindrical power, cylindrical axis, prism or base) of the VLS 1762 are programmable and controllable. The VLS 1762 may be implemented in many different ways. For example, in illustrative implementations, the VLS 1762 includes one or more of the following: an Alvarez lens pair, Jackson cross-cylinders, Humphrey lenses, a sphero-cylindrical lens pair, Risley prisms, or liquid lenses.

In the example shown in FIG. 17, the controller device 1760 also includes additional apparatus 1763 for assessing refractive aberrations or other conditions or parameters of one or both eyes of a human user.

In some cases, the additional apparatus 1763 includes apparatus for taking objective refractive measurements 1764 (i.e., measurements that do not involve feedback regarding the user's subjective visual perception). An iterative testing procedure that involves feedback regarding the user's subjective visual perceptions is performed. In some cases, the objective measurement apparatus 1764 takes measurements during each iteration of an iterative vision test. Alternatively or in addition, the variable lens system 1762 is used to improve measurements taken by the objective measurement apparatus, by optimizing focusing into the retina. In some implementations, the apparatus for objective refractive measurement 1764 comprises one or more of the following: (1) an auto-refractor, which automates a Scheiner's test with a lens and fundus camera to assess the image quality of a known source falling into the retina; (2) a Shack-Hartmann device for wavefront sensing, which analyzes the distortions of a known light pattern reflected onto a human retina and creates a wavefront map; or (3) a retroillumination system, which captures images of an eye structure while illuminating the eye structure from the rear (e.g., by reflected light).

In some cases, the additional apparatus 1763 includes relaxation apparatus 1765. The relaxation apparatus 1765 presents stimuli to either an eye being tested, the other eye, or both eyes. The stimuli tend to control the accommodation (and thus the optical power) of the user's eyes. In some cases, the relaxation apparatus includes a combination of one or more of the following (a) a lens or group of lenses, (b) actuators for moving the lens or lenses, (c) masks or other spatial light attenuators, (d) mirrors, optical fibers or other relay optics for steering light, and (e) a display screen or film for displaying images.

In some cases, an iterative vision test is performed to measure refractive aberrations (e.g., myopia, hyperopia, prism, astigmatism, spherical aberration, coma or trefoil) of the eyes of a human user. The test is performed while the controller device 1760 is positioned in front of the user's eyes. The test involves the use of one or more of the VLS 1762, apparatus for objective refractive assessment 1764 and the relaxation apparatus 1765. In some cases, the iterative vision test involves displaying images on a screen 1721 of an MDS 1720 that is releasably attached to the controller device 1760. For examples, in some cases the iterative eye test is performed in the manner described in the NETRA Patent, and the images that are displayed on an MCD screen include images that are described in the NETRA Patent. As used herein, the “NETRA Patent” means U.S. Pat. No. 87,817,871 B2, Near Eye Tool for Refractive Assessment, Vitor Pamplona et al. The NETRA Patent is incorporated herein by reference.

In some cases, a computer (e.g., onboard the MCD) analyzes data gathered during the iterative eye test and calculates refractive aberration data—that is, data indicative of one or more refractive aberrations of the eyes of a human user. The computer takes this refractive aberration data as input and outputs control signals to control one or more devices in order to compensate for the refractive aberrations. For example, in some cases, the control signals control the VLS 1762 such that the VLS 1762 corrects (compensates for) the refractive aberrations indicated by the refractive aberration data. Or, in some cases, the control signals cause visual images displayed by a screen of the MCD to be distorted in such a way as to compensate for the refractive aberrations indicated by the refractive aberration data. This distortion of images displayed by the MCD screen is sometimes called warping or pre-warping.

In some cases, the refractive aberrations are corrected (e.g., by controlling the VLS or distorting the MCD images) while a user watches visual content displayed by the MCD 1730, such as a photograph, interactive game, or virtual reality display. Thus, the user sees the visual content with corrected vision, without the need for eyeglasses or contacts.

In some cases, the refractive aberrations are corrected (e.g., by controlling the VLS or distorting the MCD images) while a user watches an augmented reality display. In the augmented reality display, images are displayed on an optical element (e.g., a half-silvered surface) that both reflects and transmits light, so that the user sees not only the augmented reality display that reflects from the optical element but also sees the light from an external scene that passes through the optical element.

This ability to detect and correct (compensate for) refractive aberrations of the human eye, without using conventional eyeglasses, is advantageous, including in virtual reality and augmented reality applications.

As noted above, the controller device 1760 is not always handheld. In some cases, the controller device is worn on the head or otherwise head-mounted. For applications in which the user is watching a long movie, or an interactive game, or a prolonged virtual reality display, it is sometimes advantageous for the controller device 1760 to be head-mounted or otherwise worn on the head, and for supplemental I/O devices that are not housed in the MCD 1720 or handheld device 1760 to be also used. For example, in some cases, the supplemental I/O devices include wireless communication modules for communicating with the MCD 1720.

In some cases, the additional apparatus 1763 onboard the controller device 1760 includes imaging apparatus 1766. The imaging apparatus 1776 includes one or more cameras and lenses. In some cases, the imaging apparatus 1766 images the retina or other parts or structures of a human eye. In some cases, the imaging apparatus 1766 is used to detect conditions of the human eye, including cataracts, retinal detachment or strabismus. In some cases, the imaging apparatus 1766 is used to measure inter-ocular distance or the orientation of the eye.

In some cases, the additional apparatus 1763 includes a set of concentric rings 1767 around each eyeport (e.g., 1108, 1109). In some cases, the concentric rings 1767 comprise active light sources, such as LEDs (light emitting diodes). In other cases, the concentric rings 1767 comprise reflective surfaces that are illuminated by light sources (such as an LED, display screen or flash) onboard the MCD 1720.

In some implementations, corneal topography is measured as follows: Concentric rings 1767 are actively illuminated (if they are active light sources, such as LEDs) or passively illuminated (if they are passive light sources, such as reflective surfaces). Light from the rings 1767 reflects off of the anterior surface of the cornea of an eye. The imaging apparatus 1766 onboard the controller device 1760 (or camera 1727 onboard the MCD 1720) captures images of the reflected light. A computer (e.g., onboard MCD 1720) analyzes these images in order to map the surface curvature of the cornea.

In some cases, the additional apparatus 1763 includes a tonometer 1768 that measures intraocular pressure of eyes of a human user. For example, in some cases, the tonometer 1768 comprises an applanation tonometer (which measures force needed to flatten an area of the cornea), such as a Goldmann tonometer or Perkins tonometer. In some cases, the tonometer 1768 comprises a dynamic contour tonometer. In some cases, the tonometer 1768 performs non-contact (e.g., air-puff) tonometry measurements.

In some cases, controller device 1760 includes relay optics 1769. The relay optics 1769 increase, decrease or shift a camera's field of view, and thereby (a) increase spatial resolution and (b) center the control components in a captured image. The increased spatial resolution facilitates optical tracking of visual features (e.g., 420) of moving control components (e.g., 406, 415, 419, 423) and increases the range (depth) of such optical tracking.

### Computers

In illustrative implementations, one or more electronic computers (e.g. 622, 1312, 1729) are programmed and specially adapted: (1) to control the operation of, or interface with, hardware components of a mobile computing device (MCD), including one or more cameras, light sources (including flashes and LEDs), screens (including display screens or capacitive touch screens), graphical user interfaces, I/O devices and wireless communication modules; (2) to control the operation of, or interface with, hardware components of a controller device, including a variable lens system, apparatus for objective refractive measurements, imaging apparatus, light sources (e.g., an array of LEDs that form concentric rings), or tonometer; (3) to analyze frames captured by the camera to detect motion of visual features, to map the motion to control signals, and to generate the control signals to control one or more operations of the MCD, including altering a display of a graphical user interface; (4) to perform any other calculation, computation, program, algorithm, computer function or computer task described or implied above; (5) to receive signals indicative of human input; (6) to output signals for controlling transducers for outputting information in human perceivable format; and (7) to process data, to perform computations, to execute any algorithm or software, and to control the read or write of data to and from memory devices. In illustrative implementations, the one or more computers are onboard the MCD. Alternatively, at least one of the computers is remote from the MCD. The one or more computers are connected to each other or to other devices either: (a) wirelessly, (b) by wired connection, or (c) by a combination of wired and wireless links

In illustrative implementations, one or more computers are programmed to perform any and all calculations, computations, programs, algorithms, computer functions and computer tasks described or implied above. For example, in some cases: (a) a machine-accessible medium has instructions encoded thereon that specify steps in a software program; and (b) the computer accesses the instructions encoded on the machine-accessible medium, in order to determine steps to execute in the program. In illustrative implementations, the machine-accessible medium comprises a tangible non-transitory medium. In some cases, the machine-accessible medium comprises (a) a memory unit or (b) an auxiliary memory storage device. For example, in some cases, a control unit in a computer fetches the instructions from memory.

In illustrative implementations, one or more computers execute programs according to instructions encoded in one or more tangible, non-transitory, machine-readable media. For example, in some cases, these instructions comprise instructions for a computer to perform any calculation, computation, program, algorithm, computer function or computer task described or implied above. For example, in some cases, instructions encoded in a tangible, non-transitory, computer-accessible medium comprise instructions for a computer to: (1) to control the operation of, or interface with, hardware components of a mobile computing device (MCD), including one or more cameras, light sources (including flashes and LEDs), screens (including display screens or capacitive touch screens), graphical user interfaces, I/O devices and wireless communication modules; (2) to control the operation of, or interface with, hardware components of a controller device, including a variable lens system, apparatus for objective refractive measurements, imaging apparatus, light sources (e.g., an array of LEDs that form concentric rings), or tonometer; (3) to analyze frames captured by the camera to detect motion of visual features, to map the motion to control signals, and to generate the control signals to control one or more operations of the MCD, including altering a display of a graphical user interface; (4) to perform any other calculation, computation, program, algorithm, computer function or computer task described or implied above; (5) to receive signals indicative of human input; (6) to output signals for controlling transducers for outputting information in human perceivable format; and (7) to process data, to perform computations, to execute any algorithm or software, and to control the read or write of data to and from memory devices.

### Network Communication

In illustrative implementations of this invention, a mobile computing device (MCD) includes a wireless communication module for wireless communication with other electronic devices in a network. The wireless communication module (e.g., module 626, 1726, 1776) includes (a) one or more antennas, (b) one or more wireless transceivers, transmitters or receivers, and (c) signal processing circuitry. The wireless communication module receives and transmits data in accordance with one or more wireless standards.

In illustrative implementations, one or more computers onboard the MCD are programmed for wireless communication over a network. For example, in some cases, one or more computers are programmed for network communication: (a) in accordance with the Internet Protocol Suite, or (b) in accordance with any industry standard for wireless communication, including IEEE 802.11 (wi-fi), IEEE 802.15 (bluetoothhigbee), IEEE 802.16, IEEE 802.20 and including any mobile phone standard, including GSM (global system for mobile communications), UMTS (universal mobile telecommunication system), CDMA (code division multiple access, including IS-95, IS-2000, and WCDMA), or LTS (long term evolution).

### Definitions

The terms “a” and “an”, when modifying a noun, do not imply that only one of the noun exists.

To compute “based on” specified data means to perform a computation that takes the specified data as an input.

Here are some non-limiting examples of a “camera”: (a) a video camera; (b) a digital camera; (c) a sensor that records images; (d) a light sensor; (e) apparatus that includes a light sensor or an array of light sensors; and (f) apparatus for gathering data about light incident on the apparatus. The term “camera” includes any computers that process data captured by the camera.

The term “comprise” (and grammatical variations thereof) shall be construed as if followed by “without limitation”. If A comprises B, then A includes B and may include other things.

The term “computer” includes any computational device that performs logical and arithmetic operations. For example, in some cases, a “computer” comprises an electronic computational device, such as an integrated circuit, a microprocessor, a mobile computing device, a laptop computer, a tablet computer, a personal computer, or a mainframe computer. In some cases, a “computer” comprises: (a) a central processing unit, (b) an ALU (arithmetic/logic unit), (c) a memory unit, and (d) a control unit that controls actions of other components of the computer so that encoded steps of a program are executed in a sequence. In some cases, a “computer” also includes peripheral units including an auxiliary memory storage device (e.g., a disk drive or flash memory), or includes signal processing circuitry. However, a human is not a “computer”, as that term is used herein.

A “control canvas” means a set of visual features, in which the presence, position or motion of certain visual features is indicative of a user command or instruction, or is used to control the operation of another device. The term “control canvas” does not imply that a canvas textile is present.

“Controller” means a device that controls one or more hardware features or operations of another device.

“Defined Term” means a term or phrase that is set forth in quotation marks in this Definitions section.

For an event to occur “during” a time period, it is not necessary that the event occur throughout the entire time period. For example, an event that occurs during only a portion of a given time period occurs “during” the given time period.

The term “e.g.” means for example.

The fact that an “example” or multiple examples of something are given does not imply that they are the only instances of that thing. An example (or a group of examples) is merely a non-exhaustive and non-limiting illustration.

“Eyeport” means a hole or opening through which a human eye looks. In some but not all cases, an eyeport surrounds a lens or other optical element, such that light which passes through the eyeport travels through the lens or other optical element.

Unless the context clearly indicates otherwise: (1) a phrase that includes “a first” thing and “a second” thing does not imply an order of the two things (or that there are only two of the things); and (2) such a phrase is simply a way of identifying the two things, respectively, so that they each can be referred to later with specificity (e.g., by referring to “the first” thing and “the second” thing later). For example, unless the context clearly indicates otherwise, if an equation has a first term and a second term, then the equation may (or may not) have more than two terms, and the first term may occur before or after the second term in the equation. A phrase that includes a “third” thing, a “fourth” thing and so on shall be construed in like manner.

The term “for instance” means for example.

As used herein, the “forehead” means the region of a human face that covers the frontal bone, including the supraorbital ridges.

“Frontal bone” means the os frontale.

“Herein” means in this document, including text, specification, claims, abstract, and drawings.

As used herein: (1) “implementation” means an implementation of this invention; (2) “embodiment” means an embodiment of this invention; (3) “case” means an implementation of this invention; and (4) “use scenario” means a use scenario of this invention.

The term “include” (and grammatical variations thereof) shall be construed as if followed by “without limitation”.

“Intensity” means any measure of or related to intensity, energy or power. For example, the “intensity” of light includes any of the following measures: irradiance, spectral irradiance, radiant energy, radiant flux, spectral power, radiant intensity, spectral intensity, radiance, spectral radiance, radiant exitance, radiant emittance, spectral radiant exitance, spectral radiant emittance, radiosity, radiant exposure or radiant energy density.

“I/O device” means an input/output device. For example, an I/O device includes any device for (a) receiving input from a human, (b) providing output to a human, or (c) both. For example, an I/O device includes a graphical user interface, keyboard, mouse, touch screen, microphone, handheld controller, display screen, speaker, or projector for projecting a visual display. Also, for example, an I/O device includes any device (e.g., button, dial, knob, slider or haptic transducer) for receiving input from, or providing output to, a human.

“Light” means electromagnetic radiation of any frequency. For example, “light” includes, among other things, visible light and infrared light. Likewise, any term that directly or indirectly relates to light (e.g., “imaging ”) shall be construed broadly as applying to electromagnetic radiation of any frequency.

“Metallics” means metallic surfaces or surfaces that are covered with metallic paint,

The term “mobile computing device” or “MCD” means a device that includes a computer, a camera, a display screen and a wireless transceiver. Non-limiting examples of an MCD include a smartphone, cell phone, mobile phone, phablet, tablet computer, laptop computer and notebook computer.

To “multiply” includes to multiply by an inverse. Thus, to “multiply” includes to divide.

The term “or” is inclusive, not exclusive. For example A or B is true if A is true, or B is true, or both A or B are true. Also, for example, a calculation of A or B means a calculation of A, or a calculation of B, or a calculation of A and B.

A parenthesis is simply to make text easier to read, by indicating a grouping of words. A parenthesis does not mean that the parenthetical material is optional or can be ignored.

The term “refractive aberration” means an optical aberration, of any order, of a refractive optical element such as a human eye. Non-limiting examples of “refractive aberration” of a human eye include myopia, hyperopia, prism (or tilt), astigmatism, secondary astigmatism, spherical aberration, coma, trefoil, and quadrafoil.

As used herein, a “set” must have at least two elements. The term “set” does not include a group with no elements and does not include a group with only one element. Mentioning a first set and a second set does not, in and of itself, create any implication regarding whether or not the first and second sets overlap (that is, intersect).

“Some” means one or more.

“Substantially” means at least ten percent. For example: (a) 112 is substantially larger than 100; and (b) 108 is not substantially larger than 100.

The term “such as” means for example.

To say that a medium has instructions encoded “thereon” means that the instructions are encoded on or in the medium.

“User interface” means an I/O device, as defined herein.

“Variable lens system” means a system of one or more lenses, the optical power of which system is adjustable.

Except to the extent that the context clearly requires otherwise, if steps in a method are described herein, then the method includes variations in which: (1) steps in the method occur in any order or sequence, including any order or sequence different than that described; (2) any step or steps in the method occurs more than once; (3) different steps, out of the steps in the method, occur a different number of times during the method, (4) any combination of steps in the method is done in parallel or serially; (5) any step or steps in the method is performed iteratively; (6) a given step in the method is applied to the same thing each time that the given step occurs or is applied to different things each time that the given step occurs; or (7) the method includes other steps, in addition to the steps described.

This Definitions section shall, in all cases, control over and override any other definition of the Defined Terms. For example, the definitions of Defined Terms set forth in this Definitions section override common usage or any external dictionary. If a given term is explicitly or implicitly defined in this document, then that definition shall be controlling, and shall override any definition of the given term arising from any source (e.g., a dictionary or common usage) that is external to this document. If this document provides clarification regarding the meaning of a particular term, then that clarification shall, to the extent applicable, override any definition of the given term arising from any source (e.g., a dictionary or common usage) that is external to this document. To the extent that any term or phrase is defined or clarified herein, such definition or clarification applies to any grammatical variation of such term or phrase, taking into account the difference in grammatical form. For example, the grammatical variations include noun, verb, participle, adjective, and possessive forms, and different declensions, and different tenses. In each case described in this paragraph, Applicant is acting as Applicant's own lexicographer.

### Variations

This invention may be implemented in many different ways. Here are some non-limiting examples:

In one aspect, this invention is a method comprising, in combination: (a) a first component of an apparatus undergoing a first movement relative to housing of the apparatus, while a surface of the apparatus is pressed against the forehead and cheeks of a human user and the apparatus is attached to a mobile computing device; (b) a first camera onboard the mobile computing device capturing images indicative of the first movement; and (c) a computer onboard the mobile computing device processing the images to recognize the first movement and, based on data indicative of the first movement, generating control signals to control, at least in part, operation of the mobile computing device. In some cases, the control signals control at least part of a display on a screen of the mobile computing device. In some cases, the control signals cause a visual feature displayed on a screen of the mobile computing device to undergo a second movement, which second movement is calculated by the computer, such that the second movement is a function of the first movement. In some cases, a second component of the apparatus has one or more visual features that: (a) are in a fixed position relative to the housing; and (b) are indicative of a path of the first movement. In some cases, the visual features are offset at a specified distance from the path. In some cases, the visual features are positioned at the beginning and end of the path, or are offset at a specified distance from the beginning and end of the path. In some cases, the screen displays images used in an assessment of refractive aberrations of an eye of the human user. In some cases, the computer outputs signals to adjust a variable lens system onboard the apparatus, such that the variable lens system compensates for at least one refractive aberration of a user's eyes. In some cases, the variable lens system compensates for at least one refractive aberration of a user's eyes while (i) visual content is displayed on the screen and (ii) light from the screen reaches the eyes of the user. In some cases, the computer outputs signals that cause the screen to display visual content that is warped by a distortion, which distortion at least partially compensates for at least one refractive aberration of an eye of the user. In some cases, the computer generates, based at least in part on data indicative of the first movement, signals that control a tonometer onboard the apparatus, which tonometer measures intraocular pressure of an eye of the user. In some cases, the computer generates, based at least in part on data indicative of the first movement, signals that control a second camera onboard the apparatus, which second camera captures visual data regarding the retina or other structures or parts of an eye of the user. In some cases, the computer processes the visual data and detects a condition or parameter of an eye of the human, which condition or parameter is not a refractive aberration. In some cases, the computer generates, based at least in part on data indicative of the first movement, signals that control a corneal topography device onboard the apparatus, which corneal topography device measures surface curvature of a cornea of an eye of the user. Each of the cases described above in this paragraph is an example of the method described in the first sentence of this paragraph, and is also an example of an embodiment of this invention that is combinable with any other feature or embodiment of this invention.

In another aspect, this invention is a system comprising, in combination: (a) apparatus which (i) includes an external curved surface that is configured to be pressed against the forehead and cheeks of a human user, (ii) includes an attachment mechanism for attaching the apparatus to a mobile computing device, and (iii) includes a first component that is configured to undergo movement relative to housing of the apparatus; and (b) a machine-readable medium having instructions encoded thereon for a computer: (i) to generate control signals that cause a first camera onboard the mobile computing device to capture images indicative of the movement, and (ii) to process the images to recognize the movement and, based on data indicative of the movement, to generate control signals to control at least, at least in part, operation of the mobile computing device. In some cases, the machine-readable medium is tangible and does not comprise a transitory signal. In some cases, the instructions encoded on the machine-readable medium include instructions for a computer to output control signals to cause a screen onboard the mobile computing device to display images used in an assessment of refractive aberrations of an eye of the human user. In some cases, the instructions encoded on the machine-readable medium include instructions for a computer to output control signals to control timing of the first camera and a light source onboard the mobile computing device, such that the emission of light by the light source and capture of images by the camera are synchronized. In some cases: (a) a second component of the apparatus has a fixed position relative to the housing; and (b) the second component has one or more visual features that are indicative of a path of the first movement. In some cases: (a) the images include data regarding a set of components of the apparatus, which set includes the first component; (b) at least some components in the set of components have a different color than the color of other components in the set; and (c) the instructions encoded on the machine-readable medium include instructions for a computer to output control signals to cause a light source onboard the mobile computing device to change, over time, color of light emitted by the light source. In some cases: (a) the images include data regarding a set of components of the apparatus, which set includes the first component; (b) at least some components in the set of components have a different color than the color of other components in the set; and (c) the instructions encoded on the machine-readable medium include instructions for a computer to change, over time, which colors are enhanced or suppressed during processing of images captured by the camera. In some cases, the instructions encoded on the machine-readable medium include instructions for the computer to output signals that cause a screen onboard the mobile computing device to display visual content that is warped by a distortion, which distortion at least partially compensates for at least one refractive aberration of an eye of the user. In some cases, the instructions encoded on the machine-readable medium include instructions for causing a tonometer onboard the apparatus to measure intraocular pressure of an eye of the user. In some cases, the instructions encoded on the machine-readable medium include instructions for causing a second camera onboard the apparatus to capture visual data regarding the retina or other structures or parts of an eye of the user. In some cases, the instructions encoded on the machine-readable medium include instructions for the computer to process the visual data and detect a condition or parameter of an eye of the human, which condition or parameter is not a refractive aberration. In some cases, the instructions encoded on the machine-readable medium include instructions for causing a corneal topography device onboard the apparatus to measure surface curvature of a cornea of an eye of the user. In some cases, the instructions encoded on the machine-readable medium include instructions for the computer to output signals to adjust a variable lens system onboard the apparatus, such that the variable lens system at least partially compensates for at least one refractive aberration of an eye of the user. In some cases, the instructions encoded on the machine-readable medium include instructions for the computer to cause the variable lens system to at least partially compensate for at least one refractive aberration of an eye of the user while (i) visual content is displayed on the screen and (ii) light from the screen reaches the eyes of the user. Each of the cases described above in this paragraph is an example of the system described in the first sentence of this paragraph, and is also an example of an embodiment of this invention that is combinable with any other feature or embodiment of this invention.

In another aspect, this invention comprises apparatus that: (a) includes an attachment mechanism for attaching the apparatus to a mobile computing device; (b) includes a first component that is configured to undergo movement relative to housing of the apparatus; (c) includes an external curved surface that is configured to be pressed against the forehead and cheeks of a human user; and (d) has a hole which extends through the apparatus, such that, when the external curved surface is pressed against the forehead and cheeks and the apparatus is attached to the mobile computing device, a view through the apparatus exists, the view being through the hole to at least a portion of a screen of the mobile computing device. In some cases: (a) a second component of the apparatus is in a fixed position relative to the housing; and (b) the second component has one or more visual features that are indicative of a path of the movement. In some cases, the visual features are offset at a specified distance from the path. In some cases: (a) the first component has a first color and the second component has a second color; and (b) the first color is different than the second color. In some cases, the first component has a specular surface. In some cases, the first component has a surface such that, when incident light from a light source strikes the surface and reflects from the surface, the intensity of light reflected by the first component is greatest in a direction toward the light source. Each of the cases described above in this paragraph is an example of the apparatus described in the first sentence of this paragraph, and is also an example of an embodiment of this invention that is combinable with any other feature or embodiment of this invention.

The above description (including without limitation any attached drawings and figures) describes illustrative implementations of the invention. However, the invention may be implemented in other ways. The methods and apparatus which are described above are merely illustrative applications of the principles of the invention. Other arrangements, methods, modifications, and substitutions by one of ordinary skill in the art are therefore also within the scope of the present invention. Numerous modifications may be made by those skilled in the art without departing from the scope of the invention. Also, this invention includes without limitation each combination and permutation of one or more of the abovementioned implementations, embodiments and features.

1. A method comprising, in combination:
(a) a first component of an apparatus undergoing a first movement relative to housing of the apparatus, while a surface of the apparatus is pressed against the forehead and cheeks of a human user and the apparatus is attached to a mobile computing device;
(b) a first camera onboard the mobile computing device capturing images indicative of the first movement; and
(c) a computer onboard the mobile computing device processing the images to recognize the first movement and, based on data indicative of the first movement, generating control signals to control, at least in part, operation of the mobile computing device.

2. The method of claim 1, wherein the control signals control at least part of a display on a screen of the mobile computing device.

3. The method of claim 1, wherein the control signals cause a visual feature displayed on a screen of the mobile computing device to undergo a second movement, which second movement is calculated by the computer, such that the second movement is a function of the first movement.

4. The method of claim 1, wherein a second component of the apparatus has one or more visual features that:
(a) are in a fixed position relative to the housing; and
(b) are indicative of a path of the first movement.

5. The method of claim 4, wherein the visual features are offset at a specified distance from the path.

6. The method of claim 4, wherein the visual features are positioned at the beginning and end of the path, or are offset at a specified distance from the beginning and end of the path.

7. The method of claim 2, wherein the screen displays images used in an assessment of refractive aberrations of an eye of the human user.

8. A system comprising, in combination:
(a) apparatus which
(i) includes an external curved surface that is configured to be pressed against the forehead and cheeks of a human user,
(ii) includes an attachment mechanism for attaching the apparatus to a mobile computing device, and
(iii) includes a first component that is configured to undergo movement relative to housing of the apparatus; and
(b) a computer programmed:
(i) to generate control signals that cause a first camera onboard the mobile computing device to capture images indicative of the movement, and
(ii) to process the images to recognize the movement and, based on data indicative of the movement, to generate control signals to control at least, at least in part, operation of the mobile computing device.

9. (canceled)

10. The system of claim 8, wherein the computer is programmed to output control signals to cause a screen onboard the mobile computing device to display images used in an assessment of refractive aberrations of an eye of the human user.

11. The system of claim 8, wherein the computer is programmed to output control signals to control timing of the first camera and a light source onboard the mobile computing device, such that the emission of light by the light source and capture of images by the camera are synchronized.

12. The system of claim 8, wherein:
(a) a second component of the apparatus has a fixed position relative to the housing; and
(b) the second component has one or more visual features that are indicative of a path of the first movement.

13. The system of claim 8, wherein:
(a) the images include data regarding a set of components of the apparatus, which set includes the first component;
(b) at least some components in the set of components have a different color than the color of other components in the set; and
(c) the computer is programmed to output control signals to cause a light source onboard the mobile computing device to change, over time, color of light emitted by the light source.

14. The system of claim 8, wherein:
(a) the images include data regarding a set of components of the apparatus, which set includes the first component;
(b) at least some components in the set of components have a different color than the color of other components in the set; and
(c) the computer is programmed to change, over time, which colors are enhanced or suppressed during processing of images captured by the camera.

15. An apparatus that:
(a) includes an attachment mechanism for attaching the apparatus to a mobile computing device;
(b) includes a first component that is configured to undergo movement relative to housing of the apparatus;
(c) includes an external curved surface that is configured to be pressed against the forehead and cheeks of a human user; and
(d) has a hole which extends through the apparatus, such that, when the external curved surface is pressed against the forehead and cheeks and the apparatus is attached to the mobile computing device, a view through the apparatus exists, the view being through the hole to at least a portion of a screen of the mobile computing device.

16. The apparatus of claim 15, wherein:
(a) a second component of the apparatus is in a fixed position relative to the housing; and
(b) the second component has one or more visual features that are indicative of a path of the movement.

17. The apparatus of claim 16, wherein the visual features are offset at a specified distance from the path.

18. The apparatus of claim 15, wherein:
(a) the first component has a first color and the second component has a second color; and
(b) the first color is different than the second color.

19. The apparatus of claim 15, wherein the first component has a specular surface.

20. The apparatus of claim 15, wherein the first component has a surface such that, when incident light from a light source strikes the surface and reflects from the surface, the intensity of light reflected by the first component is greatest in a direction toward the light source.

21. The method of claim 2, wherein the computer outputs signals that cause the screen to display visual content that is warped by a distortion, which distortion at least partially compensates for at least one refractive aberration of an eye of the user.

22. (canceled)

23. (canceled)

24. (canceled)

25. (canceled)

26. (canceled)

27. (canceled)

28. (canceled)

29. (canceled)

30. (canceled)
